(19) United States
(12) Patent Application Publication
(10) Pub. No.: US 2023/0000843 A1
(43) Pub. Date: Jan. 5, 2023

(54) MEDICINAL COGNITIVE TREATMENTS

(71) Applicant: ACTINOGEN MEDICAL LIMITED, Sydney, New South Wales (AU)

(72) Inventors: Tamara Diane MILLER, Sydney, New South Wales (AU); John William KETELBEY, Sydney, New South Wales (AU)

(21) Appl. No.: 17/764,922

(22) PCT Filed: Sep. 30, 2020
(86) PCT No.: PCT/AU2020/051043

§ 371 Date: Mar. 29, 2022

(30) Foreign Application Priority Data
Sep. 30, 2019 (AU) 2019903681

(51) Int. Cl. A61K 31/439; A61K 31/444; A61K 31/506; A61K 31/497; A61P 25/28

(52) U.S. Cl. CPC A61K 31/439; A61K 31/444; A61K 31/506; A61K 31/497; A61P 25/28

(57) ABSTRACT

The present disclosure generally relates to a method of improving cognition and/or treating cognitive decline in a cognitively healthy subject comprising administering to the cognitively healthy subject a therapeutically effective amount of a compound of Formula I, or a pharmaceutically acceptable salt, solvate or prodrug thereof, as described herein.

### FIELD

The present disclosure generally relates to the field of pharmaceutical treatments for improving cognition and/or treating decline in cognition in cognitively healthy subjects. The present disclosure also relates to a method of treating and/or preventing cognitive decline in cognitively healthy subjects. The present disclosure also relates to a method of improving cognitive performance in cognitively healthy subjects.

### BACKGROUND

Change in cognitive performance as a normal process of aging has been documented in scientific literature. Some cognitive abilities, such as vocabulary, are resistant to brain aging and may even improve with age. Other cognitive abilities, such as conceptual reasoning, memory, and processing speed, can gradually decline with age. Such changes in cognitive abilities associated with aging are commonly referred to as “age-related cognitive decline”.

With an increasingly aging population, age-related cognitive decline is emerging as a prevalent health and social issue. Indeed, cognitive decline is among the most feared aspects of growing old. It is also the most costly, in terms of the financial, personal and societal burdens.

It is well accepted in the scientific community that cognitive ability can be divided into specific cognitive domains, being processing speed, attention, memory, language, visuospatial abilities/construction, and executive functioning/reasoning.

Processing speed refers to the speed with which cognitive activities are performed as well as the speed of motor responses, and this fluid ability begins to decline in the third decade of life and continues throughout the lifespan. Attention refers to the ability to concentrate and focus on specific stimuli, with more noticeable age-related declines seen in more complex attention tasks, such as selective and divided attention. Memory includes both declarative (explicit) memory and non-declarative (implicit) memory, though it is the declarative memory in which age-related decline is more commonly witnessed. Language is a complex cognitive domain composed of both crystallised and fluid cognitive abilities, and while general vocabulary remains relatively unchanged with age, visual confrontation naming, or the ability to see a common object and name it, declines dramatically from about 70 years of age. Visual construction skills, which involves the ability to put together individual parts to make a coherent whole, also typically declines over time. In contrast, visuospatial abilities, which includes the ability to recognise familiar objects such as household items or faces, typically remains relatively intact with age. Lastly, executive functioning/reasoning refers to capacities that allow a person to successfully engage in independent, appropriate, purposive, and self-serving behaviour, and research has shown that concept formation, abstraction, and mental flexibility decline with age, particularly after 70 years of age.

The reasons behind age-related cognitive decline remain to be fully elucidated. From a physiological perspective, it is understood that grey matter volume begins to decrease after 20 years of age, with the majority of atrophy occurring in the prefrontal cortex. Even more significant decreases in volume occur in the white matter as well as its functioning. Declines in neurotransmitter levels may also contribute to age-related cognitive decline.

These physiological reasons, however, do not explain the variability in age-related cognitive decline amongst individuals. To this extent, it is estimated that approximately 60% of age-related cognitive decline may be attributed to genetics, though it is also believed that medical illness, psychological factors, and sensory deficits (e.g., vision and hearing impairment) can also accelerate age-related cognitive decline.

Irrespective of the pathophysiology, although age-related cognitive decline may not impair a person's ability to perform daily activities, it is undisputed that normal, age-related cognitive decline in a healthy, aging individual can result in subtle declines in complex functional abilities. A good example of this, is the ability to drive, which poses as a safety issue for not only the aging population, but those that may be inadvertently affected.

It is believed that participation in certain activities, building cognitive reserve, and engaging in cognitive retraining may all be approaches to delay age-related cognitive decline. Tentative associations between exercise, diet, alcohol, sleep and social activity have also been linked to age-related cognitive decline. However, these mechanisms influence age-related cognitive decline in a variable manner amongst different individuals.

Accordingly, there remains a need for alternative and effective treatments for improving cognition in cognitively healthy subjects and/or treating and/or preventing cognitive decline in cognitively healthy subjects, particularly in aging, cognitively healthy individuals.

### SUMMARY

The subject matter of the present disclosure is predicated in part on the surprising discovery that an azabicyclothiophenyl compound of Formula I as described herein can improve cognition and/or treat cognitive decline in cognitively healthy subjects.

Accordingly, in a first aspect, there is provided a method of preventing and/or treating cognitive decline in a cognitively healthy subject, comprising administering to the cognitively healthy subject a therapeutically effective amount of a compound of Formula I, or a pharmaceutically acceptable salt, solvate or prodrug thereof:

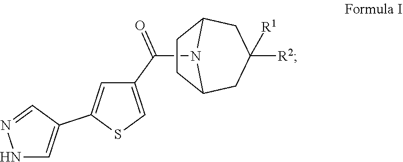

wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR³, —NR³R⁴, —COR³, —CO₂R³, —CONR³R⁴, —NR³COR⁴, —SO₂R³, —SO₂NR³R⁴, and —NR³SO₂R⁴; wherein R³ and R⁴ are independently selected from the group consisting of hydrogen, C₁₋₆alkyl, 3-7-membered carbocyclyl and 3-7-membered heterocyclyl; wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl.

In a further aspect, there is provided a method of preventing and/or treating cognitive decline in a cognitively healthy subject, comprising administering to the cognitively healthy subject a therapeutically effective amount of a compound of Formula I, or a pharmaceutically acceptable salt, solvate or prodrug thereof:

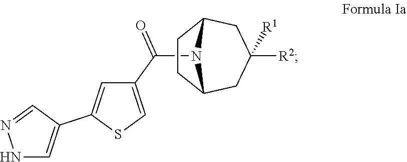

wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR³, —NR³R⁴, —COR³, —CO₂R³, —CONR³R⁴, —NR³COR⁴, —SO₂R³, —SO₂NR³R⁴, and —NR³SO₂R⁴; wherein R³ and R⁴ are independently selected from the group consisting of hydrogen, C₁₋₆alkyl, 3-7-membered carbocyclyl and 3-7-membered heterocyclyl; wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl.

In some embodiments, wherein R¹ and R² are each independently selected from the group consisting of hydrogen, halogen, 3-10-membered carbocyclyl, 3-10-membered heterocyclyl, —OH, —CN, and —NH₂; and wherein if present, each 3-10-membered carbocyclyl and 3-10-membered heterocyclyl may be further substituted with one or more substituents selected from the group consisting of hydrogen, halogen, —OH, —CN, —CF₃, —NH₂, and C₁₋₆alkyl.

In some embodiments, R¹ and R² are each independently selected from the group consisting of hydrogen, halogen, 6-membered carbocyclyl, 6-membered heterocyclyl, —OH, —CN, and —NH₂; and wherein if present, each 6-membered carbocyclyl and 6-membered heterocyclyl may be further substituted with one or more substituents selected from the group consisting of hydrogen, halogen, —OH, —CN, —CF₃, —NH₂, and C₁₋₆alkyl.

In some embodiments, R¹ is selected from the group consisting of hydrogen, halogen, —OH, —CN, —CF₃, —NH₂, and C₁₋₆alkyl, and R² is selected from the group consisting of:

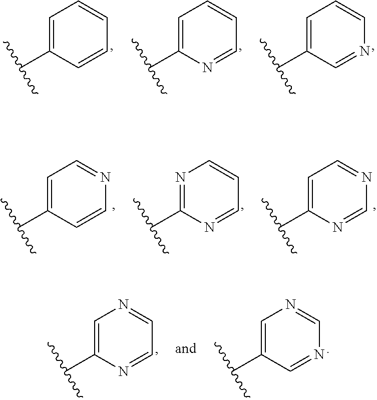

In some embodiments, the compound of Formula I is selected from the group consisting of:

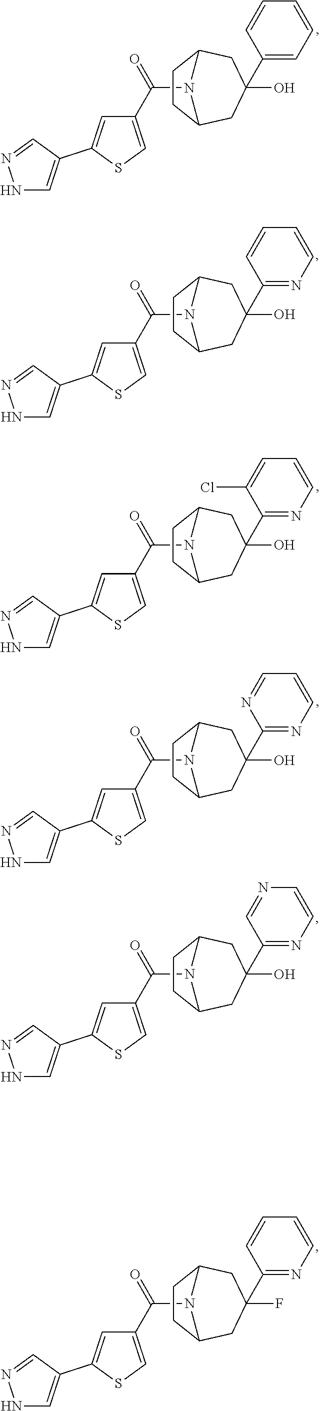 [SMILES: O=C(c1csc(-c2cn[nH]c2)c1)N1C2CCC1CC(O)(c1ccccc1)C2 ; O=C(c1csc(-c2cn[nH]c2)c1)N1C2CCC1CC(O)(c1ccccn1)C2 ; O=C(c1csc(-c2cn[nH]c2)c1)N1C2CCC1CC(O)(c1ncccc1Cl)C2 ; O=C(c1csc(-c2cn[nH]c2)c1)N1C2CCC1CC(O)(c1ncccn1)C2 ; O=C(c1csc(-c2cn[nH]c2)c1)N1C2CCC1CC(O)(c1cnccn1)C2 ; O=C(c1csc(-c2cn[nH]c2)c1)N1C2CCC1CC(F)(c1ccccn1)C2]

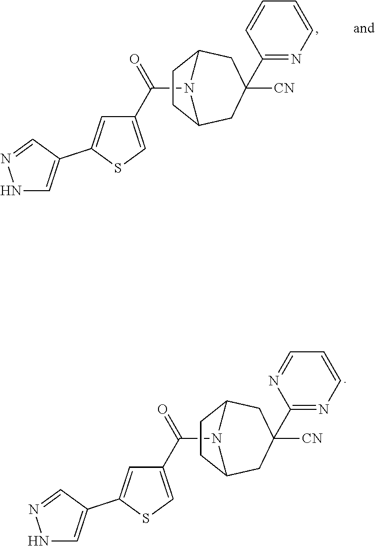 [SMILES: N#CC1(c2ccccn2)CC2CCC(C1)N2C(=O)c1csc(-c2cn[nH]c2)c1 ; N#CC1(c2ncccn2)CC2CCC(C1)N2C(=O)c1csc(-c2cn[nH]c2)c1]

In some embodiments, the compound of Formula I is:

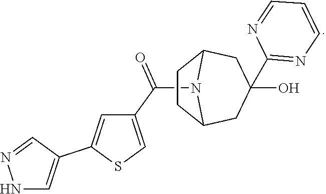 [SMILES: O=C(c1csc(-c2cn[nH]c2)c1)N1C2CCC1CC(O)(c1ncccn1)C2]

In some embodiments, the compound of Formula Ia is:

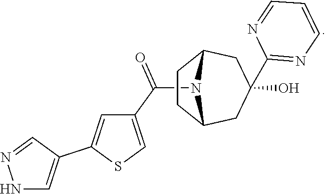 [SMILES: O=C(c1csc(-c2cn[nH]c2)c1)N1[C@@H]2CC[C@H]1C[C@](O)(c1ncccn1)C2]

In some embodiments, the cognitively healthy subject is not suffering from another, diagnosed medical condition that is associated with the central nervous system (CNS).

In some embodiments, the cognitively healthy subject has an increased risk of cognitive decline.

In some embodiments, the increased risk of cognitive decline is provided by a risk factor selected from the group consisting of an increased risk of stroke, the presence of genetic markers associated with cognitive decline, a family history of cognitive decline, environmental factors associated with cognitive decline, societal factors associated with cognitive decline, external factors associated with cognitive decline, and side effects associated with therapeutic treatment.

In some embodiments, the administration of a compound of Formula I, or a pharmaceutically acceptable salt, solvate or prodrug thereof, provides improved cognitive ability of the treated subject relative to a non-treated subject.

In some embodiments, the improved cognitive ability of the treated subject is determinable by cognitive testing.

In some embodiments, the administration of a compound of Formula I, or a pharmaceutically acceptable salt, solvate or prodrug thereof, provides an improvement of psychomotor function of the treated subject relative to a non-treated subject.

In some embodiments, the improvement of psychomotor function is at least partial restoration of motor function.

In some embodiments, the improvement in psychomotor function is provided by an improvement in simple reaction time.

In some embodiments, the administration of a compound of Formula I, or a pharmaceutically acceptable salt, solvate or prodrug thereof, provides an improvement of visual attention of the treated subject relative to a non-treated subject.

In some embodiments, the improvement in visual attention is provided by an improvement in choice retention time.

In some embodiments, the administration of a compound of Formula I, or a pharmaceutically acceptable salt, solvate or prodrug thereof, provides an improvement of learning and memory of the treated subject relative to a non-treated subject.

In some embodiments, the improvement in learning and memory is provided by an improvement in visual recognition learning.

In some embodiments, the administration of a compound of Formula I, or a pharmaceutically acceptable salt, solvate or prodrug thereof, provides an improvement of working memory of the treated subject relative to a non-treated subject.

In some embodiments, the administration of a compound of Formula I, or a pharmaceutically acceptable salt, solvate or prodrug thereof, provides an improvement of visual associate memory of the treated subject relative to a non-treated subject.

In some embodiments, the cognitively healthy subject has age-related cognitive decline.

In some embodiments, the cognitively healthy subject is of at least 1, at least 5, at least 10, at least 20, at least 30, at least 40, at least 50, at least 60, at least 70, at least 80, at least 90, or at least 100 years of age.

In some embodiments, the cognitively healthy subject has a low IQ.

In some embodiments, the cognitively healthy subject is between about 1 and about 20 years of age.

In some embodiments, the cognitively healthy subject is male.

In some embodiments, the compound of Formula I, or a pharmaceutically acceptable salt, solvate or prodrug thereof, is administered in amount so as to deliver a total daily dosage of from about 10 to about 40 mg of Formula I.

In some embodiments, the compound of Formula I, or a pharmaceutically acceptable salt, solvate or prodrug thereof, is administered as a single once-daily dosage or a twice-daily dosage.

In some embodiments, the compound of Formula I, or a pharmaceutically acceptable salt, solvate or prodrug thereof, is administered as a twice-daily dosage of 10 mg per dosage.

In some embodiments, the compound of Formula I, or a pharmaceutically acceptable salt, solvate or prodrug thereof, is administered orally.

In some embodiments, the compound of Formula I, or a pharmaceutically acceptable salt, solvate or prodrug thereof, is administered with food.

In a further aspect, there is provided use of a compound of Formula I, or a pharmaceutically acceptable salt, solvate or prodrug thereof:

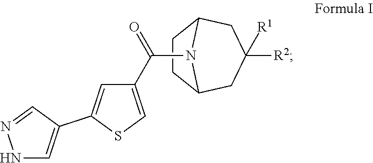

wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR³, —NR³R⁴, —COR³, —CO₂R³, —CONR³R⁴, —NR³COR⁴, —SO₂R³, —SO₂NR³R⁴, and —NR³SO₂R⁴; wherein R³ and R⁴ are independently selected from the group consisting of hydrogen, C₁₋₆alkyl, 3-7-membered carbocyclyl and 3-7-membered heterocyclyl; wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl; in the manufacture of a medicament for the prevention and/or treatment of cognitive decline in a cognitively healthy subject.

In some embodiments, the compound of Formula I, or a pharmaceutically acceptable salt, solvate or prodrug thereof, is a compound of Formula Ia:

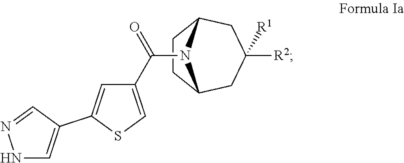

wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR³, —NR³R⁴, —COR³, —CO₂R³, —CONR³R⁴, —NR³COR⁴, —SO₂R³, —SO₂NR³R⁴, and —NR³SO₂R⁴; wherein R³ and R⁴ are independently selected from the group consisting of hydrogen, C₁₋₆alkyl, 3-7-membered carbocyclyl and 3-7-membered heterocyclyl; wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl.

In some embodiments, the compound of Formula Ia is:

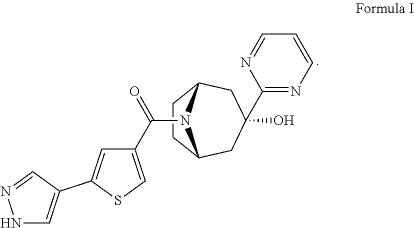 [SMILES: O=C(c1csc(-c2cn[nH]c2)c1)N1[C@@H]2CC[C@H]1C[C@](O)(c1ncccn1)C2]

In a further aspect, there is provided use of a compound of Formula I, or a pharmaceutically acceptable salt, solvate or prodrug thereof:

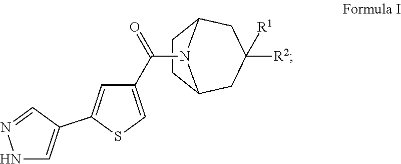

wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR³, —NR³R⁴, —COR³, —CO₂R³, —CONR³R⁴, —NR³COR⁴, —SO₂R³, —SO₂NR³R⁴, and —NR³SO₂R⁴; wherein R³ and R⁴ are independently selected from the group consisting of hydrogen, C₁₋₆alkyl, 3-7-membered carbocyclyl and 3-7-membered heterocyclyl; wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl; for the prevention and/or treatment of cognitive decline in a cognitively healthy subject.

In some embodiments, the compound of Formula I, or a pharmaceutically acceptable salt, solvate or prodrug thereof, is a compound of Formula Ia:

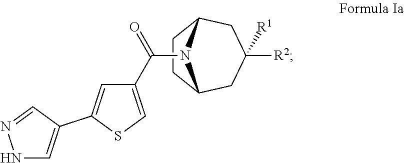

wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR³, —NR³R⁴, —COR³, —CO₂R³, —CONR³R⁴, —NR³COR⁴, —SO₂R³, —SO₂NR³R⁴, and —NR³SO₂R⁴; wherein R³ and R⁴ are independently selected from the group consisting of hydrogen, C₁₋₆alkyl, 3-7-membered carbocyclyl and 3-7-membered heterocyclyl; wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl.

In some embodiments, the compound of Formula Ia is:

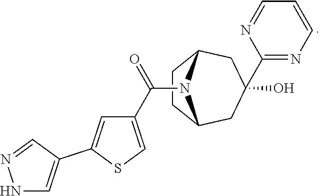 [SMILES: O=C(c1csc(-c2cn[nH]c2)c1)N1[C@@H]2CC[C@H]1C[C@](O)(c1ncccn1)C2]

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### BRIEF DESCRIPTION OF THE DRAWINGS

Whilst it will be appreciated that a variety of embodiments of the disclosure may be utilised, in the following, we describe a number of examples of the disclosure with reference to the following drawings: the abbreviation “LSMeans” refers to “Least Squares Means”.

FIG. 1A(1) shows Means and 95% confidence intervals of the test scores of all the subjects who took Xanamem™, which is a compound of Formula Ia as described herein, vs. all the subjects who took placebo in the Detection Test; and FIG. 1A(2) shows LSMeans and 95% confidence intervals of the same.

FIG. 1B(1) shows Means and 95% confidence intervals of the test scores of all the female subjects who took Xanamem™ vs. all the female subjects who took placebo in the Detection Test; and FIG. 1B(2) shows LSMeans and 95% confidence intervals of the same.

FIG. 1C(1) shows Means and 95% confidence intervals of the test scores of all the male subjects who took Xanamem™ vs. all the male subjects who took placebo in the Detection Test; and FIG. 1C(2) shows LSMeans and 95% confidence intervals of the same.

FIG. 1D shows a linear plot of mean (+SD) Detection Test results over visit by treatment group.

FIG. 2A(1) shows Means and 95% confidence intervals of the test scores of all the subjects who took Xanamem™ vs. all the subjects who took placebo in the Identification Test; and FIG. 2A(2) shows LSMeans and 95% confidence intervals of the same.

FIG. 2B(1) shows Means and 95% confidence intervals of the test scores of all the female subjects who took Xanamem™ vs. all the female subjects who took placebo in the Identification Test; and FIG. 2B(2) shows LSMeans and 95% confidence intervals of the same.

FIG. 2C(1) shows Means and 95% confidence intervals of the test scores of all the male subjects who took Xanamem™ vs. all the male subjects who took placebo in the Identification Test; and FIG. 2C(2) shows LSMeans and 95% confidence intervals of the same.

FIG. 2D shows a linear plot of mean (±SD) Identification Test results over visit by treatment group.

FIG. 3A(1) shows Means and 95% confidence intervals of the test scores of all the subjects who took Xanamem™ vs. all the subjects who took placebo in the One Card Learning Test; and FIG. 3A(2) shows LSMeans and 95% confidence intervals of the same.

FIG. 3B(1) shows Means and 95% confidence intervals of the test scores of all the female subjects who took Xanamem™ vs. all the female subjects who took placebo in the One Card Learning Test; and FIG. 3B(2) shows LSMeans and 95% confidence intervals of the same.

FIG. 3C(1) shows Means and 95% confidence intervals of the test scores of all the male subjects who took Xanamem™ vs. all the male subjects who took placebo in the One Card Learning Test; and FIG. 3C(2) shows LSMeans and 95% confidence intervals of the same.

FIG. 3D shows a linear plot of mean (±SD) One Card Learning Test results over visit by treatment group.

FIG. 4A(1) shows Means and 95% confidence intervals of the test scores of all the subjects who took Xanamem™ vs. all the subjects who took placebo in the One Back Working Memory Test; FIG. 4A(2) shows LSMeans and 95% confidence intervals of the same.

FIG. 4B(1) shows Means and 95% confidence intervals of the test scores of all the female subjects who took Xanamem™ vs. all the female subjects who took placebo in the One Back Working Memory Test; and FIG. 4B(2) shows LSMeans and 95% confidence intervals of the same.

FIG. 4C(1) shows Means and 95% confidence intervals of the test scores of all the male subjects who took Xanamem™ vs. all the male subjects who took placebo in the One Back Working Memory Test; and FIG. 4C(2) exhibits LSMeans and 95% confidence intervals of the same.

FIG. 4D shows a linear plot of mean (±SD) One Back Test results over visit by treatment group.

FIG. 5A(1) shows Means and 95% confidence intervals of the test scores of all the subjects who took Xanamem™ vs. all the subjects who took placebo in the Continuous Paired Associate Learning Test; and FIG. 5A(2) exhibits LSMeans and 95% confidence intervals of the same.

FIG. 5B(1) shows Means and 95% confidence intervals of the test scores of all the female subjects who took Xanamem™ vs. all the female subjects who took placebo in the Continuous Paired Associate Learning Test; and FIG. 5B(2) exhibits LSMeans and 95% confidence intervals of the same.

FIG. 5C(1) shows Means and 95% confidence intervals of the test scores of all the male subjects who took Xanamem™ vs. all the male subjects who took placebo in the Continuous Paired Associate Learning Test; and FIG. 5C(2) shows LSMeans and 95% confidence intervals of the same.

FIG. 5D shows a linear plot of mean (±SD) Continuous Paired Associated Learning Test results over visit by treatment group.

FIG. 6A(1) shows Means and 95% confidence intervals of the test scores of all the subjects who took Xanamem™ vs. all the subjects who took placebo in the Continuous Paired Associate Learning-Delayed Test; and FIG. 6A(2) shows LSMeans and 95% confidence intervals of the same.

FIG. 6B(1) shows Means and 95% confidence intervals of the test scores of all the female subjects who took Xanamem™ vs. all the female subjects who took placebo in the Continuous Paired Associate Learning-Delayed Test; and FIG. 6B(2) exhibits LSMeans and 95% confidence intervals of the same.

FIG. 6C(1) shows Means and 95% confidence intervals of the test scores of all the male subjects who took Xanamem™ vs. all the male subjects who took placebo in the Continuous Paired Associate Learning-Delayed Test; and FIG. 6C(2) exhibits LSMeans and 95% confidence intervals of the same.

FIG. 6D shows a linear plot of mean (±SD) Continuous Paired Associate Learning-Delayed Test results over visit by treatment group.

FIG. 7 shows a linear plot of mean (±SD) Plasma Xanamem™ concentrations (ng/mL) over visit by treatment group.

### DETAILED DESCRIPTION

### General Definitions

Unless specifically defined otherwise, all technical and scientific terms used herein shall be taken to have the same meaning as commonly understood by one of ordinary skill in the art (e.g., chemistry, biochemistry, medicinal chemistry, microbiology and the like).

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

As used herein, the term about, unless stated to the contrary, refers to +/−20%, typically +/−10%, typically +/−5%, of the designated value.

As used herein, the terms “a”, “an” and “the” include both singular and plural aspects, unless the context clearly indicates otherwise.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be clearly understood that, although a number of prior art publications are referred to herein, this reference does not constitute an admission that any of these documents forms part of the common general knowledge in the art, in Australia or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will prevail. In addition, the materials, methods, and examples are illustrative only and not intended to be limiting.

As used herein, the term “treating” (or “treat”, “treatment” etc.) includes a reduction, alleviation and/or elimination of one or more symptoms associated with a specific disorder or condition. Such symptoms may be correlated with cognitive decline in cognitively healthy subjects, which includes age-related cognitive decline in cognitively healthy subjects. For example, as used herein, the phrase “treating cognitive decline” includes improving, reducing, alleviating and/or eliminating symptoms associated with cognitive decline, relative to the symptoms prior to treatment.

As used herein, the term “preventing” (or “prevention”) includes prophylaxis of the specific disorder or condition. For example, as used herein, the phrase “preventing cognitive decline” refers to preventing the onset or duration of the symptoms associated with cognitive decline in cognitively healthy subjects, which includes age-related cognitive decline in cognitively healthy subjects. In some embodiments, the phrase “preventing cognitive decline” refers to slowing or halting the progression of cognitive decline. In some embodiments, the phrase “preventing cognitive decline” refers to delaying or preventing the onset of the symptoms of the cognitive decline. Prevention may be absolute (such that no cognitive decline occurs), or may be effective only in some individuals, to some extent, or for a limited amount of time.

As used herein, the term “halogen” means fluorine, chlorine, bromine, or iodine.

As used herein, the term “alkyl” encompasses both straight-chain (i.e., linear) and branched-chain hydrocarbon groups. Examples of alkyl groups include, but are not limited to, methyl, ethyl, n-propyl, iso-propyl, n-butyl, t-butyl, i-butyl, sec-butyl, pentyl, and hexyl groups. In one example, the alkyl group is of one to six carbon atoms (i.e. C₁₋₆alkyl).

As used herein, the term “alkoxy” refers to the group —O-alkyl, where “alkyl” is as described above. Examples of alkoxy groups include, but are not limited to, methoxy, ethoxy, propoxy, and butoxy groups. In one example, the alkoxy group is of one to six carbon atoms (i.e. —O—C₁₋₆alkyl).

As used herein, the term “alkenyl” refers to both straight and branched chain unsaturated hydrocarbon groups with at least one carbon-carbon double bond. Examples of alkenyl groups include, but are not limited to, ethenyl, propenyl, butenyl, pentenyl, and hexenyl groups. In one example, the alkenyl group is of two to six carbon atoms (i.e. C₂₋₆alkenyl).

As used herein, the term “alkynyl” refers to both straight and branched chain unsaturated hydrocarbon groups with at least one carbon-carbon triple bond. Examples of alkynyl groups include, but are not limited to, ethynyl, propynyl, butynyl, pentynyl, and hexynyl groups. In one example, the alkynyl group is of two to six carbon atoms (i.e. C₂₋₆alkynyl).

As used herein, the term “haloalkyl” refers to an alkyl group having at least one halogen substituent, where “alkyl” and “halogen” are as described above. Similarly, the term “dihaloalkyl” means an alkyl group having two halogen substituents, and the term “trihaloalkyl” means an alkyl group having three halogen substituents. Examples of haloalkyl groups include fluoromethyl, chloromethyl, bromomethyl, iodomethyl, fluoropropyl, and fluorobutyl groups. Examples of dihaloalkyl groups include difluoromethyl and difluoroethyl groups. Examples of trihaloalkyl groups include trifluoromethyl and trifluoroethyl groups. In one example, the haloalkyl group is of one to six carbon atoms (i.e. C₁₋₆haloalkyl).

As used herein, the term “oxyhaloalkyl” refers to the group —O-haloalkyl, where “haloalkyl” is as described above. Examples of —O-haloalkoxy groups include —O-fluoromethyl, —O-chloromethyl, —O-bromomethyl, —O-iodomethyl, —O-fluoropropyl, and —Ofluorobutyl groups. In one example, the oxyhaloalkyl group is of one to six carbon atoms (i.e. —O—C₁₋₆haloalkyl).

As used herein, the term “carbocyclyl” refers to an aromatic or non-aromatic cyclic group of carbon atoms. A carbocyclyl group may, for example, be monocyclic or polycyclic (i.e. bicyclic, tricyclic). A polycyclic carbocyclyl group may contain fused rings. In one example, the carbocyclyl group is of three to ten carbon atoms (i.e. C₃₋₁₀carbocyclyl). In one example, the carbocyclyl group is of three to seven carbon atoms (i.e. C₃₋₇carbocyclyl). Examples of monocyclic non-aromatic carbocyclyl groups include cyclopropyl, cyclobutyl, cyclopentyl, cyclopentenyl, cyclohexyl, cyclohexenyl, cycloheptyl, and cyclooctyl groups. Aromatic carbocyclyl groups include phenyl and napthalenyl.

As used herein, the term “heterocyclyl” refers to an aromatic or non-aromatic cyclic group which is analogous to a carbocyclic group, but in which from one to three of the carbon atoms is/are replaced by one or more heteroatoms independently selected from nitrogen, oxygen, or sulfur. A heterocyclyl group may, for example, be monocyclic or polycyclic (e.g. bicyclic). A polycyclic heterocyclyl may for example contain fused rings. In a bicyclic heterocyclyl group there may be one or more heteroatoms in each ring, or heteroatoms only in one of the rings. A heteroatom may be N, O, or S. Heterocyclyl groups containing a suitable nitrogen atom include the corresponding N-oxides. In one example, the heterocyclyl group is of three to ten atoms (i.e. 3-10-membered heterocyclyl). In one example, the heterocyclyl group is of three to seven atoms (i.e. 3-7-membered heterocyclyl). Examples of monocyclic non-aromatic heterocyclyl groups include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl. Examples of bicyclic heterocyclyl groups in which one of the rings is non-aromatic include dihydrobenzofuranyl, indanyl, indolinyl, isoindolinyl, tetrahydroisoquinolinyl, tetrahydroquinolyl, and benzoazepanyl. Examples of monocyclic aromatic heterocyclyl groups (also referred to as monocyclic heteroaryl groups) include furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl. Examples of bicyclic aromatic heterocyclyl groups (also referred to as bicyclic heteroaryl groups) include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridyl, pyridopyrimidinyl, isoquinolinyl, and benzohydroxazole.

### Cognitive Decline

The subject matter of the present disclosure is predicated in part on the surprising discovery that a compound of Formula I improves cognition and/or treats cognitive decline in a cognitively healthy subject. Accordingly, in one aspect, there is provided a method of preventing and/or treating cognitive decline in a cognitively healthy subject.

As used herein, “cognitive decline” refers to the deterioration, or loss, of cognitive ability. The skilled person will be aware that there are a multitude of reasons as to why a subject may present with cognitive decline. Further, it is understood that cognitive decline is a normal physiological process that occurs, to some extent, in all subjects.

Although not limited to, cognitive decline has been demonstrated in an aging population. As used herein, “age-related cognitive decline” refers to the deterioration, or loss, of cognitive ability associated with increasing age in an otherwise cognitively healthy subject. As used herein, the term “subject” may be used interchangeably with the terms “patient” and “individual”. Also as used herein, the term “subject” refers to any organism that is susceptible to a disease or condition. In some embodiments, the subject is a mammal. In some embodiments, the subject is a human.

### Cognitively Healthy Subject

Such deterioration, or loss, of cognitive ability, may be evident in an otherwise cognitively healthy subject irrespective of whether that subject is suffering from another medical condition. In some embodiments, the subject is not suffering from another medical condition. In some embodiments, the subject is suffering from another medical condition. The other medical condition may be diagnosed or undiagnosed. The other medical condition may not have an association with cognitive decline or age-related cognitive decline. That is, the other medical condition may not be indicative of, more prevalent in, or linked to, cognitive decline or age related cognitive decline. In some embodiments, the subject is suffering from another, diagnosed medical condition that is not associated with cognitive decline or age related cognitive decline.

Where the subject is not suffering from another, diagnosed medical condition that is associated with cognitive decline, the subject is considered cognitively healthy. That is, as used herein, the term “cognitively healthy” refers to a subject not having been diagnosed with another medical condition associated with cognitive decline. As would be understood by the person skilled in the art, the natural physiological process of cognitive decline is consistent with an otherwise cognitively healthy subject. Similarly, the natural aging process, and the natural age-related cognitive decline, is consistent with an otherwise cognitively healthy subject. That is, a subject may experience, or suffer from, age related cognitive decline and yet be considered “cognitively healthy”.

Other medical conditions that may be associated with cognitive decline include medical conditions that primarily affect, or are highly associated with, the central nervous system (CNS). In some embodiments, the subject is not suffering from another, undiagnosed medical condition that is associated with the central nervous system (CNS). In some embodiments, the subject is not suffering from another, diagnosed medical condition that is associated with the central nervous system (CNS), for example mild cognitive impairment or Alzheimer's disease (AD). It is therefore understood that, in some embodiments, a “cognitively healthy” subject is a subject that is not suffering from another, undiagnosed or diagnosed, medical condition that is associated with the central nervous system (CNS).

In further embodiments, the cognitively healthy subject may not have a disorder associated with a substantially elevated level of cortisol, such as dementia. Dementias include, but are not limited to Alzheimer's disease, multi-infarct dementia, dementia with Lewy bodies, fronto-temporal dementia (including Pick's disease), progressive supranuclear palsy, Korsakoff's syndrome, Biswanger's disease, HIV-associated dementia, Creutzfeldt-Jakob disease (CJD), multiple sclerosis, motor neurone disease, Parkinson's disease, Huntington's disease, Niemann-Pick disease type C, normal pressure hydrocephalus, and Down's syndrome.

The cognitively healthy subject may have a cortisol level (in nmol/L) of less than about 450, 440, 430, 420, 410, 400, 390, 380, 370, 360, 250, 340, 330, 320, 310, 300, 290, 280, 270, 260, 250, 240, 230, 220, 210, or 200. The cognitively healthy subject may have a cortisol level (in nmol/L) of at least about 150, 160, 170, 180, 190, 200, 210, 220, 230, 240, 250, 260, 270, 280, 290, 300, 310, 320, 330, 340, or 350. The cognitively healthy subject may have a cortisol level (in nmol/L) in a range provided by any two of these upper and/or lower levels. For example, the cognitively healthy subject may have a cortisol level (in nmol/L) in a range between about 150 and 400, 170 and 350, or 200 and 330. The cortisol level may be measured from the plasma or serum of a subject. The cortisol level may represent an average daily or weekly measurement.

### Subjects

The cognitively healthy subject may be a human or an animal. For example, the methods as described herein may be used for human or veterinary applications. In addition to humans, domesticated animals may include dogs, cats, horses, cattle, sheep, pigs, or birds, for example.

### Age and Gender of Subjects

It is understood that cognitive decline varies greatly amongst different subjects. Such variations include, but are not limited to, age of onset and severity of symptoms. As used herein, “age of onset” refers to the subject's age in which symptoms of cognitive decline first present. It is generally understood that there is a link between increasing age, or “aging”, of the subject and the first presentation of the symptoms of cognitive decline, particularly age-related cognitive decline. That is, increasing age, or aging, is believed to coincide with the first presentation of the symptoms of cognitive decline. However, the age in which the symptoms of cognitive decline first present varies greatly amongst different subjects. For example, the symptoms of cognitive decline may first present in a subject being of about 5 years, about 10 years, about 15 years, about 20 years, about 30 years, about 40 years, about 50 years, about 60 years, about 70 years, about 80 years, about 90 years, about 100 years, about 110 years, or about 120 years of age. A subject may present with a low intelligence quotient, or “IQ”, but be otherwise cognitively healthy. Methods for assessing the IQ of an individual are known in the art. As used herein, the term “low IQ” refers to an IQ score of less than about 85. In some embodiments, the subject has an IQ score of less than about 85, 75, 65, 55, 45, 35, or about 25. It is foreseeable that the method disclosed herein would be beneficial in a subject having a low IQ. In some embodiments, the age (in years) of the subject is at least about 5, 10, 15, 20, 30, 40, 50, 60, 70, 80, 90, or 100. In some embodiments, the age of the subject (in years) is less than about 90, 80, 70, 60, 50, 40, 30, 20, or 10. The age of the subject may be in a range between any two of these upper and/or lower values.

Cognitive decline may also be dependent upon the subject's gender. An earlier age of onset and an increase in severity of symptoms of cognitive decline can occur in males. In some embodiments, the subject is a male. In some embodiments, the subject is a cognitively healthy male subject. In some embodiments, the subject is a cognitively healthy male subject of at least 40 years of age. it will be appreciated that females also remain at risk of cognitive decline. In some embodiments, the subject is a female. In some embodiments, the subject is a cognitively healthy female subject. In some embodiments, the subject is a cognitively healthy female subject of at least 40 years of age.

Subjects with Increased Risk of Cognitive Decline

It will also be understood that the method disclosed herein may find application in the prevention and/or reduction of cognitive decline in a subject that is at increased risk of cognitive decline, but is otherwise a cognitively healthy subject. “Increased risk” of cognitive decline is used herein to refer to a subject that may have a higher likelihood of experiencing cognitive decline, or may have a higher likelihood of experiencing early-onset cognitive decline, or may have a higher likelihood of experiencing more severe cognitive decline. In some embodiments, the subject has an increased risk of cognitive decline. In some embodiments, the subject has an increased risk of age-related cognitive decline. In some embodiments, the subject has an increased risk of experiencing early-onset cognitive decline. In some embodiments, the subject has an increased risk of experiencing severe cognitive decline.

The person skilled in the art will appreciate that there are a number of factors that may result in a subject having an increased risk of cognitive decline. Factors that may result in a subject having an increased risk of cognitive decline include, but are not limited to, increased risk of stroke, the presence of particular genetic markers, a family history of cognitive decline, suffering from particular medical conditions associated with cognitive decline, and/or environmental factors that are associated with cognitive decline, and/or societal factors that are associated with cognitive decline, and/or external factors that are associated with cognitive decline. In some embodiments, the subject has an increased risk of stroke. In some embodiments, the subject possesses a genetic marker associated with cognitive decline (e.g. apolipoprotein E, APOE, associated with AD). In some embodiments, the subject has a family history of cognitive decline. In some embodiments, the subject is suffering from a medical condition associated with cognitive decline. In some embodiments, the subject is exposed to environmental factors that are associated with cognitive decline. In some embodiments, the subject is exposed to societal factors that are associated with cognitive decline. Such societal factors include, but are not limited to, stress, family break-down, physical abuse, mental abuse, alcohol and/or substance abuse, and sleep deprivation. In some embodiments, the subject is exposed to external factors that are associated with, or may lead to, cognitive decline. Such external factors include, but are not limited to, recreational/professional hobby/sporting activities that result in concussion, physical stress, or mental stress. In some embodiments, the subject is exposed to therapeutic treatment, wherein the therapeutic treatment (e.g., chemotherapy, anaesthesia) is associated with side effects that include cognitive decline. In some embodiments, the subject is exposed to chemotherapy, wherein the chemotherapy is associated with side effects that include cognitive decline. In some embodiments, the subject is exposed to anaesthesia, wherein the anaesthesia is associated with side effects that include cognitive decline.

### Enhancement of Cognitive Ability

It will also be understood that the method disclosed herein may be beneficial in enhancing cognitive performance or ability in a cognitively healthy subject. As used herein, “enhancing cognitive ability” refers to an increase in cognitive ability compared to a subject's previous ability (i.e., before being administered a compound of Formula I, or a pharmaceutically acceptable salt, solvate or prodrug thereof). In some embodiments, the subject experiences enhanced cognitive ability. Such agents that are known to improve cognition are referred to in the art as “noortropic”. In some embodiments, the compound of Formula I, or a pharmaceutically acceptable salt, solvate or prodrug thereof, is a nootropic agent. In such circumstances, the subject may or may not be experiencing cognitive decline. The enhancement of cognitive ability may be experienced in relation to one or more facets of cognition including, for example, psychomotor function, visual attention, learning and memory, working memory, and visual associate memory. Such cognitive abilities may be measured using various techniques known in the field, such as those described below (e.g. Cogstate Battery Tests). In some embodiments, a subject experiences enhanced psychomotor function. In some embodiments, a subject experiences enhanced visual attention. In some embodiments, a subject experiences enhanced learning and memory. In some embodiments, a subject experiences enhanced working memory. In some embodiments, a subject experiences enhanced visual associate memory.

### Methods of Assessing Cognitive Ability

There are several tests through which the cognitive ability of a subject may be assessed. Accordingly, based on the results of these tests, it is possible to assess a subject's cognitive decline.

The person skilled in the art will appreciate that there exists a variety of tests that can be employed to accurately assess the cognitive state, and similarly the cognitive decline, of a subject. Such tests include, but are not limited to, Cogstate Battery Tests, Cambridge Neuropsychological Test Automated Battery (CANTAB), Intelligence Quotient (IQ) Test, Kohs Block Design Test, Miller Analogies Test, Otis-Lennon School Ability Test (OLSAT), Raven's Progressive Matrices, Stanford-Binet Intelligence Scales, Wechsler Intelligence Scale for Children (WISC), Wonderlic Test, Porteus Maze Test, Pimsleur Language Aptitude Battery, Knox Cubes, Draw-a-Person Test, Mini-Mental State Exam (MMSE), the Alzheimer's Disease Assessment Scale-Cognitive Subscale (ADAS-Cog, ADCOMs, Rey Auditory Visual Learning Test (RAVLT), NTB and NPI), and CDR Computerized Assessment System. Cogstate Battery Tests include, for example, Detection test, Identification test, One Card Learning test, One Back Working Memory test, and the Continuous Paired Associate Learning test. Each test is designed to assess various parameters that may be attributed to the cognitive ability of a subject. Accordingly, in some embodiments the administration of a compound of Formula I, or a pharmaceutically acceptable salt, solvate or prodrug thereof, provides improved cognitive testing (Cogstate Test Battery) of the treated subject relative to a non-treated subject. An improvement in cognitive testing, as determined by the Cogstate Test Battery results, may also be considered from a summation of each individual test results (e.g., Detection test, Identification test, One Card Learning test, One Back Working Memory test, and the Continuous Paired Associate Learning test).

In some examples, any one or more cognitive tests measuring reaction time improvement may provide an improvement of at least about (log 10, milliseconds) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.20. In some examples, any one or more cognitive tests may provide a “p value” probability measurement from a treated subject group of less than about 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001. In some examples, the cognitive testing may provide a Cohen's “d-value” standardised difference between two means (i.e. treated and untreated subject) of at least about 0.1, 0.2, 0.3, 0.4, 0.5, 0.6, 0.7, or 0.8. In some examples, the cognitive testing may provide a “p-value” and “d-value” according to any combination of individual embodiments thereof as described herein.

In some embodiments, the administration of a compound of Formula I, or a pharmaceutically acceptable salt, solvate or prodrug thereof, provides an improvement of psychomotor function of the treated subject relative to a non-treated subject. Psychomotor function is evidenced by an improvement in simple reaction time, and is measured in speed of performance (Log₁₀ milliseconds). As will be appreciated by the person skilled in the art, psychomotor function is critical in a subject's ability to execute both gross and fine motor skills. Accordingly, psychomotor function is an essential component of physical skills including, for example, movement, coordination, manipulation, dexterity, grace, strength, and speed. It is therefore foreseeable that the method described herein may find particular application in improving psychomotor function in a cognitively healthy subject. In some embodiments, the administration of a compound of Formula I, or a pharmaceutically acceptable salt, solvate or prodrug thereof, provides an improvement of psychomotor function in a cognitively healthy subject. In some embodiments, the administration of a compound of Formula I, or a pharmaceutically acceptable salt, solvate or prodrug thereof, provides an improvement in physical skill of a cognitively healthy subject. In some embodiments, the administration of a compound of Formula I, or a pharmaceutically acceptable salt, solvate or prodrug thereof, provides an improvement of gross motor skills in a cognitively healthy subject. In some embodiments, the administration of a compound of Formula I, or a pharmaceutically acceptable salt, solvate or prodrug thereof, provides an improvement of fine motor skills in a cognitively healthy subject.

From here, it is foreseeable that the method described herein may also find particular application in assisting a subject in regaining psychomotor function. A loss of psychomotor function may result for any number of reasons including, but not limited to, accident and/or injury, a stroke, or other medical condition. Such a loss of psychomotor skills may affect any one or more areas of the subject's body including, but not limited to, the limbs. Accordingly, the administration of a compound of Formula I, or a pharmaceutically acceptable salt, solvate or prodrug thereof, may assist to restore psychomotor function. The term “restore psychomotor function” is used herein to mean a return, to some extent, of previous motor function. It is therefore foreseeable that a subject having suffered from a stroke, and experiencing a subsequent loss in motor skills, may benefit from the administration of a compound of Formula I, or a pharmaceutically acceptable salt, solvate or hydrate thereof. A subject having suffered from a stroke may have lost motor function in the limbs, such that the ability to walk is compromised. In some embodiments, the administration of a compound of Formula I, or a pharmaceutically acceptable salt, solvate or prodrug thereof, restores motor skills in a cognitively healthy subject. In some embodiments, the administration of a compound of Formula I, or a pharmaceutically acceptable salt, solvate or prodrug thereof, restores motor skills in a cognitively healthy subject that had previously lost motor function due to suffering from a stroke. In some embodiments, the administration of a compound of Formula I, or a pharmaceutically acceptable salt, solvate or prodrug thereof, restores motor skills of a limb in a cognitively healthy subject. In some embodiments, the administration of a compound of Formula I, or a pharmaceutically acceptable salt, solvate or prodrug thereof, restores motor skills such as to facilitate walking in a cognitively healthy subject.

In some embodiments, the administration of a compound of Formula I, or a pharmaceutically acceptable salt, solvate or prodrug thereof, provides an improvement of psychomotor function speed of performance. The psychomotor function speed improvement may be measured between the treated subjects relative to non-treated subjects. In some examples, a psychomotor function speed improvement may be provided by a log 10 reaction time (milliseconds) of less than about 2.60. 2.59, 2.58, 2.57, 2.56, 2.55, 2.54, 2.53, 2.52, 2.51, or 2.50. In some examples, a psychomotor function speed improvement may be at least about (log 10, milliseconds) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.20. In some examples, the psychomotor function testing has a p value probability measurement from a treated subject group of less than about 0.5, 0.4, 0.3, 0.2, 0.1, or 0.09. In some examples, the psychomotor function testing may provide a Cohen's d-value standardised difference between two means (i.e. treated and untreated subject) of at least about 0.1, 0.2, 0.3, 0.4, 0.5, 0.6, or 0.7. In some examples, the psychomotor function testing may provide a “p-value” and “d-value” according to any combination of individual embodiments thereof, such as a p-value less than about 0.09 with a d-value effect size of greater than about 0.7.

In some embodiments, the administration of a compound of Formula I, or a pharmaceutically acceptable salt, solvate or prodrug thereof, provides an improvement of visual attention of the treated subject relative to a non-treated subject. Visual attention is evidenced by an improvement in choice retention time, and is measured in speed of performance (Log₁₀ milliseconds). In some embodiments, the improvement in visual attention is evidenced by an improvement in choice retention time. In some embodiments, the improvement in visual attention is assessed by the Cogstate Identification Test. In some embodiments, the administration of a compound of Formula I, or a pharmaceutically acceptable salt, solvate or prodrug thereof, provides an improvement of visual attention speed of performance of the treated subject relative to a non-treated subject. The visual attention speed improvement may be measured between the treated subjects relative to non-treated subjects. In some examples, a visual attention speed improvement may be provided by a log 10 reaction time (milliseconds) of less than about 2.80, 2.79, 2.78, 2.77, 2.76, 2.75, 2.74, 2.73, 2.72, 2.71, or 2.70. In some examples, a visual attention speed improvement may be at least about (log 10, milliseconds) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.20. In some examples, the visual attention testing has a p value probability measurement from a treated subject group of less than about 0.5, 0.4, 0.3, 0.2, 0.1, or 0.09, 0.08, 0.07, 0.06, or 0.05. In some examples, the visual attention may provide a Cohen's d-value standardised difference between two means (i.e. treated and untreated subject) of at least about 0.1, 0.2, 0.3, 0.4, 0.5, or 0.6. In some examples, the visual attention testing may provide a “p-value” and “d-value” according to any combination of individual embodiments thereof, such as a p-value less than about 0.05 with a d-value effect size of greater than about 0.6.

In some embodiments, the administration of a compound of Formula I, or a pharmaceutically acceptable salt, solvate or prodrug thereof, provides an improvement of learning and memory of the treated subject relative to a non-treated subject. Learning and memory is evidenced by an improvement in visual recognition learning, and is measured in accuracy of performance (arcsine proportion correct). In some embodiments, the improvement in learning and memory is evidenced by an improvement in visual recognition learning. In some embodiments, the improvement in learning and memory is assessed by the Cogstate One Card Learning Test. In some embodiments, the administration of a compound of Formula I, or a pharmaceutically acceptable salt, solvate or prodrug thereof, provides an improvement of learning and memory accuracy of proportion (i.e., arcsine proportion correct) of at least about X % of the treated subject relative to a non-treated subject. The learning and memory accuracy improvement may be measured between the treated subjects relative to non-treated subjects. In some examples, a learning and memory accuracy testing may be provided by an arcsine accuracy of at least about 1.0, 1.01, 1.02, 1.03, 1.04, 1.05, 1.06, 1.07, 1.08, 1.09. or 1.10. In some examples, the learning and memory accuracy testing has a p value probability measurement from a treated subject group of less than about 0.9. In some examples, the visual attention may provide a Cohen's d-value standardised difference between two means (i.e. treated and untreated subject) of at least about 0.1, 0.15, or 0.19. In some examples, the visual attention testing may provide a “p-value” and “d-value” according to any combination of individual embodiments thereof, such as a p-value less than about 0.9 with a d-value effect size of greater than about 0.15.

In some embodiments, the administration of a compound of Formula I, or a pharmaceutically acceptable salt, solvate or prodrug thereof, provides an improvement of working memory of the treated subject relative to a non-treated subject. In some embodiments, the improvement in working memory is assessed by the Cogstate One Back Working Memory Test, and is measured in number of errors. In some embodiments, the administration of a compound of Formula I, or a pharmaceutically acceptable salt, solvate or prodrug thereof, provides an improvement of working memory number of errors of at least about X % of the treated subject relative to a non-treated subject. The working memory improvement may be measured between the treated subjects relative to non-treated subjects. In some examples, a working memory test may be provided by a log 10 reaction time of less than about 2.90, 2.89, 2.88, 2.87, 2.86, 2.85, 2.84, 2.83, 2.82, 2.81, or 2.80. In some examples, a working memory test improvement may be at least about (log 10, milliseconds) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.20. In some examples, the working memory testing has a p value probability measurement from a treated subject group of less than about 0.5, 0.4, 0.3, 0.2, 0.1, or 0.09, 0.08, 0.07, 0.06, 0.05, 0.04, 0.03, 0.02, or 0.01. In some examples, the working memory testing may provide a Cohen's d-value standardised difference between two means (i.e. treated and untreated subject) of at least about 0.1, 0.2, 0.3, 0.4, 0.5, 0.6, 0.7, or 0.8. In some examples, the visual attention testing may provide a “p-value” and “d-value” according to any combination of individual embodiments thereof, such as a p-value less than about 0.01 with a d-value effect size of greater than about 0.8.

In some embodiments, the administration of a compound of Formula I, or a pharmaceutically acceptable salt, solvate or prodrug thereof, provides an improvement of visual associate memory of the treated subject relative to a non-treated subject. In some embodiments, visual associate memory is assessed by the Cogstate Continuous Paired Associate Learning Test, and is measured in number of errors. The visual associate memory improvement may be measured between the treated subjects relative to non-treated subjects. In some examples, a visual associate memory testing may be provided by an error score out of 30 of less than 25, 24, 23, 22, 21, 20, 19, 18, 17, 16, 15, 14, 13, 12, 11, or 10. In some examples, a visual associate memory testing may be provided by an improvement in error score of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, the visual associate memory testing has a p value probability measurement from a treated subject group of less than about 0.5. In some examples, the visual associate memory testing may provide a Cohen's d-value standardised difference between two means (i.e. treated and untreated subject) of at least about 0.1, 0.2, 0.3, or 0.4. In some examples, the visual associate memory testing may provide a “p-value” and “d-value” according to any combination of individual embodiments thereof, such as a p-value less than about 0.5 with a d-value effect size of greater than about 0.4.

### Compound of Formula I

It has been surprisingly found that a compound of Formula I is useful in a method of preventing and/or treating cognitive decline in a cognitively healthy subject. Embodiments of the methods and uses described herein comprise administering a compound of Formula I, or a pharmaceutically acceptable salt, solvate or prodrug thereof.

As used herein, a compound of Formula I has the following chemical structure:

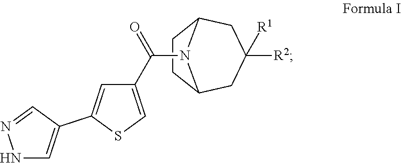

wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR³, —NR³R⁴, —COR³, —CO₂R³, —CONR³R⁴, —NR³COR⁴, —SO₂R³, —SO₂NR³R⁴, and —NR³SO₂R⁴.

In some embodiments, R¹ is hydrogen. In some embodiments, R¹ is halogen. In some embodiments, R¹ is chlorine. In some embodiments, R¹ is fluorine. In some embodiments, R¹ is bromine. In some embodiments, R¹ is iodine. In some embodiments, R¹ is C₁₋₆alkyl. In some embodiments, R¹ is —O—C₁₋₆alkyl. In some embodiments, R¹ is C₁₋₆haloalkyl. In some embodiments, R¹ is —O—C₁₋₆haloalkyl. In some embodiments, R¹ is C₂₋₆alkenyl. In some embodiments, R¹ is C₂₋₆alkynyl. In some embodiments, R¹ is 3-10-membered carbocyclyl. In some embodiments, R¹ is a 6-membered carbocyclyl. In some embodiments, R¹ is a 5-membered carbocyclyl. In some embodiments, R¹ is 3-10-membered heterocyclyl. In some embodiments, R¹ is a 6-membered heterocyclyl. In some embodiments, R¹ is a 5-membered heterocyclyl. In some embodiments, R¹ is —CN. In some embodiments, R¹ is —CF₃. In some embodiments, R¹ is —OR³. In some embodiments, R¹ is —SR³. In some embodiments, R¹ is —NR³R⁴. In some embodiments, R¹ is —COR³. In some embodiments, R¹ is —CO₂R³. In some embodiments, R¹ is —CONR³R⁴. In some embodiments, R¹ is —NR³COR⁴. In some embodiments, R¹ is —SO₂R³. In some embodiments, R¹ is —SO₂NR³R⁴. In some embodiments, R¹ is —NR³SO₂R⁴.

In some embodiments, R² is hydrogen. In some embodiments, R² is halogen. In some embodiments, R² is chlorine. In some embodiments, R² is fluorine. In some embodiments, R² is bromine. In some embodiments, R² is iodine. In some embodiments, R² is C₁₋₆alkyl. In some embodiments, R² is —O—C₁₋₆alkyl. In some embodiments, R² is C₁₋₆haloalkyl. In some embodiments, R² is —O—C₁₋₆haloalkyl. In some embodiments, R² is C₂₋₆alkenyl. In some embodiments, R² is C₂₋₆alkynyl. In some embodiments, R² is 3-10-membered carbocyclyl. In some embodiments, R² is a 6-membered carbocyclyl. In some embodiments, R² is a 5-membered carbocyclyl. In some embodiments, R² is 3-10-membered heterocyclyl. In some embodiments, R² is a 6-membered heterocyclyl. In some embodiments, R² is a 5-membered heterocyclyl. In some embodiments, R² is —CN. In some embodiments, R² is —CF₃. In some embodiments, R² is —OR³. In some embodiments, R² is —SR³. In some embodiments, R² is —NR³R⁴. In some embodiments, R² is —COR³. In some embodiments, R² is —CO₂R³. In some embodiments, R² is —CONR³R⁴. In some embodiments, R² is —NR³COR⁴. In some embodiments, R² is —SO₂R³. In some embodiments, R² is —SO₂NR³R⁴. In some embodiments, R² is —NR³SO₂R⁴.

If present, each 3-10-membered carbocyclyl and 3-10-membered heterocyclyl may be further substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶.

In some embodiments, the 3-10-membered carbocyclyl is substituted with one or more halogen substituents. In some embodiments, the 3-10-membered carbocyclyl is substituted with one or more C₁₋₆alkyl substituents. In some embodiments, the 3-10-membered carbocyclyl is substituted with one or more —O—C₁₋₆alkyl substituents. In some embodiments, the 3-10-membered carbocyclyl is substituted with one or more C₁₋₆haloalkyl substituents. In some embodiments, the 3-10-membered carbocyclyl is substituted with one or more —O—C₁₋₆haloalkyl substituents. In some embodiments, the 3-10-membered carbocyclyl is substituted with one or more C₂₋₆alkenyl substituents. In some embodiments, the 3-10-membered carbocyclyl is substituted with one or more C₂₋₆alkynyl substituents. In some embodiments, the 3-10-membered carbocyclyl is substituted with one or more —CN substituents. In some embodiments, the 3-10-membered carbocyclyl is substituted with one or more —CF₃ substituents. In some embodiments, the 3-10-membered carbocyclyl is substituted with one or more —OR⁵ substituents. In some embodiments, the 3-10-membered carbocyclyl is substituted with one or more —SR⁵ substituents. In some embodiments, the 3-10-membered carbocyclyl is substituted with one or more —COR⁵ substituents. In some embodiments, the 3-10-membered carbocyclyl is substituted with one or more halogen substituents. In some embodiments, the 3-10-membered carbocyclyl is substituted with one or more —CO₂R⁵ substituents. In some embodiments, the 3-10-membered carbocyclyl is substituted with one or more —CONR⁵R⁶ substituents. In some embodiments, the 3-10-membered carbocyclyl is substituted with one or more —NR⁵COR⁶ substituents. In some embodiments, the 3-10-membered carbocyclyl is substituted with one or more —SO₂R⁵ substituents. In some embodiments, the 3-10-membered carbocyclyl is substituted with one or more —SO₂NR⁵R⁶ substituents. In some embodiments, the 3-10-membered carbocyclyl is substituted with one or more —NR⁵SO₂R⁶ substituents.

In some embodiments, R¹ is a 6-membered carbocyclyl and is substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶. In some embodiments, R¹ is a 5-membered carbocyclyl and is substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶.

In some embodiments, the 3-10-membered heterocyclyl is substituted with one or more halogen substituents. In some embodiments, the 3-10-membered heterocyclyl is substituted with one or more C₁₋₆alkyl substituents. In some embodiments, the 3-10-membered heterocyclyl is substituted with one or more —O—C₁₋₆alkyl substituents. In some embodiments, the 3-10-membered heterocyclyl is substituted with one or more C₁₋₆haloalkyl substituents. In some embodiments, the 3-10-membered heterocyclyl is substituted with one or more —O—C₁₋₆haloalkyl substituents. In some embodiments, the 3-10-membered heterocyclyl is substituted with one or more C₂₋₆alkenyl substituents. In some embodiments, the 3-10-membered heterocyclyl is substituted with one or more C₂₋₆alkynyl substituents. In some embodiments, the 3-10-membered heterocyclyl is substituted with one or more —CN substituents. In some embodiments, the 3-10-membered heterocyclyl is substituted with one or more —CF₃ substituents. In some embodiments, the 3-10-membered heterocyclyl is substituted with one or more —OR⁵ substituents. In some embodiments, the 3-10-membered heterocyclyl is substituted with one or more —SR⁵ substituents. In some embodiments, the 3-10-membered heterocyclyl is substituted with one or more —COR⁵ substituents. In some embodiments, the 3-10-membered heterocyclyl is substituted with one or more halogen substituents. In some embodiments, the 3-10-membered heterocyclyl is substituted with one or more —CO₂R⁵ substituents. In some embodiments, the 3-10-membered heterocyclyl is substituted with one or more —CONR⁵R⁶ substituents. In some embodiments, the 3-10-membered heterocyclyl is substituted with one or more —NR⁵COR⁶ substituents. In some embodiments, the 3-10-membered heterocyclyl is substituted with one or more —SO₂R⁵ substituents. In some embodiments, the 3-10-membered heterocyclyl is substituted with one or more —SO₂NR⁵R⁶ substituents. In some embodiments, the 3-10-membered heterocyclyl is substituted with one or more —NR⁵SO₂R⁶ substituents.

In some embodiments, R² is a 6-membered carbocyclyl and is substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶. In some embodiments, R² is a 5-membered carbocyclyl and is substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶.

If present, each R³ and R⁴ are independently selected from the group consisting of hydrogen, C₁₋₆alkyl, 3-7-membered carbocyclyl and 3-7-membered heterocyclyl.

In some embodiments, R³ is hydrogen. In some embodiments, R³ is C₁₋₆alkyl. In some embodiments, R³ is 3-7-membered carbocyclyl. In some embodiments, R³ is 3-7-membered carbocyclyl. In some embodiments, R³ is a 5-membered carbocyclyl. In some embodiments R³ is a 6-membered carbocyclyl.

In some embodiments, R⁴ is hydrogen. In some embodiments, R⁴ is C₁₋₆alkyl. In some embodiments, R⁴ is 3-7-membered carbocyclyl. In some embodiments, R⁴ is 3-7-membered carbocyclyl. In some embodiments, R⁴ is a 5-membered carbocyclyl. In some embodiments R⁴ is a 6-membered carbocyclyl.

If present, each R⁵ and R⁶ are independently selected from the group consisting of hydrogen and C₁₋₆alkyl.

In some embodiments, R⁵ is hydrogen. In some embodiments, R⁵ is C₁₋₆alkyl.

In some embodiments, R⁶ is hydrogen. In some embodiments, R⁶ is C₁₋₆alkyl.

In some embodiments, R¹ and R² are each independently selected from the group consisting of hydrogen, halogen, 3-10-membered carbocyclyl, 3-10-membered heterocyclyl, —OH, —CN, and —NH₂. If present, each 3-10-membered carbocyclyl and 3-10-membered heterocyclyl may be further substituted with one or more substituents selected from the group consisting of hydrogen, halogen, —OH, —CN, —CF₃, —NH₂, and C₁₋₆alkyl.

In some embodiments, R¹ and R² are each independently selected from the group consisting of hydrogen, halogen, 6-membered carbocyclyl, 6-membered heterocyclyl, —OH, —CN, and —NH₂. If present, each 6-membered carbocyclyl and 6-membered heterocyclyl may be further substituted with one or more substituents selected from the group consisting of hydrogen, halogen, —OH, —CN, —CF₃, —NH₂, and C₁₋₆alkyl.

In some embodiments, R¹ is selected from the group consisting of hydrogen, halogen, —OH, —CN, —CF₃, —NH₂, and C₁₋₆alkyl, and R² is independently selected from the group consisting of:

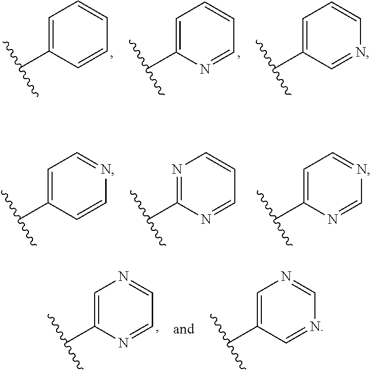

In some embodiments, R¹ is hydrogen and R² is independently selected from the group consisting of:

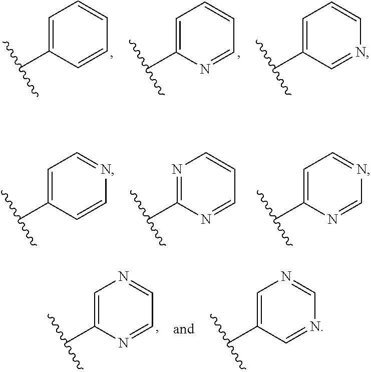

In some embodiments, R¹ is halogen and R² is independently selected from the group consisting of:

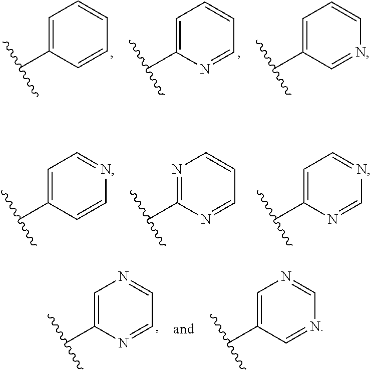

In some embodiments, R¹ is —OH and R² is independently selected from the group consisting of:

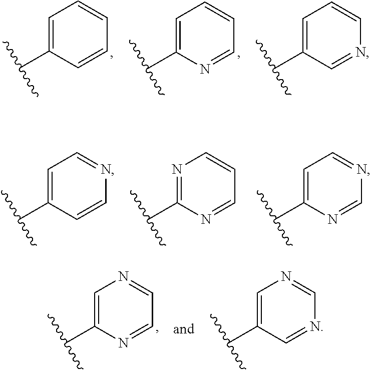

In some embodiments, R¹ is —CN and R² is independently selected from the group consisting of:

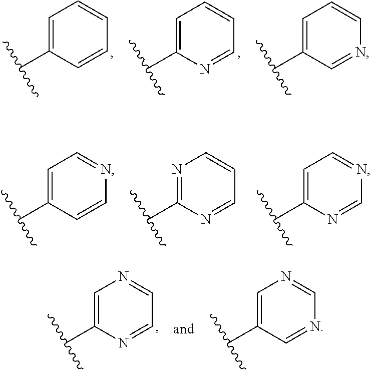

In some embodiments, R¹ is —CF₃ and R² is independently selected from the group consisting of:

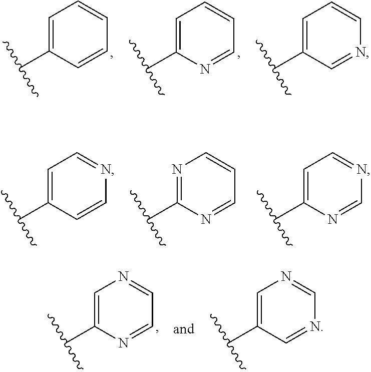

In some embodiments, R¹ is —NH₂ and R² is independently selected from the group consisting of:

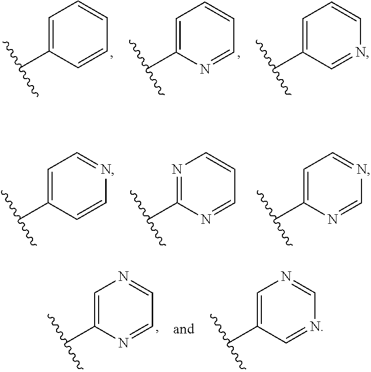

In some embodiments, R¹ is C₁₋₆alkyl and R² is independently selected from the group consisting of:

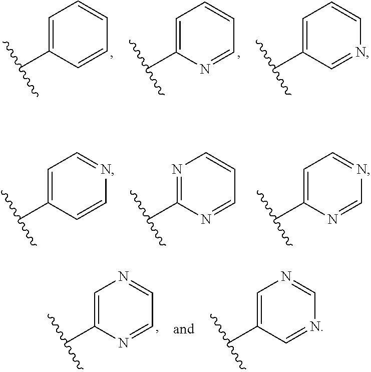

In some embodiments, R¹ is chlorine and R² is independently selected from the group consisting of:

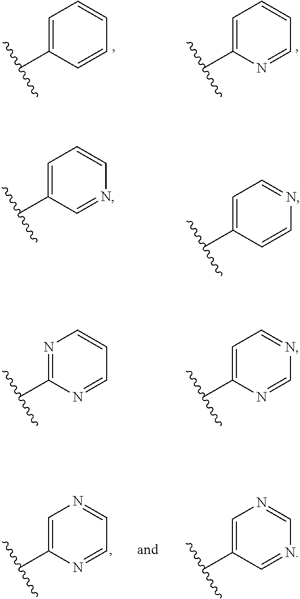

In some embodiments, R¹ is bromine and R² is independently selected from the group consisting of:

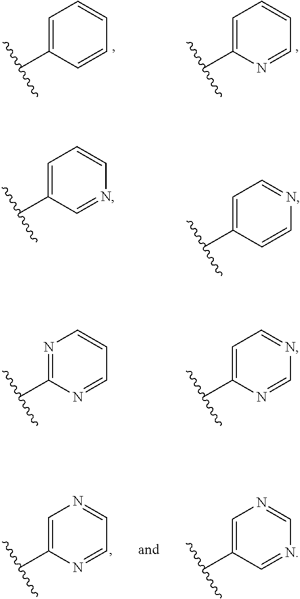

In some embodiments, R¹ is fluorine and R² is independently selected from the group consisting of:

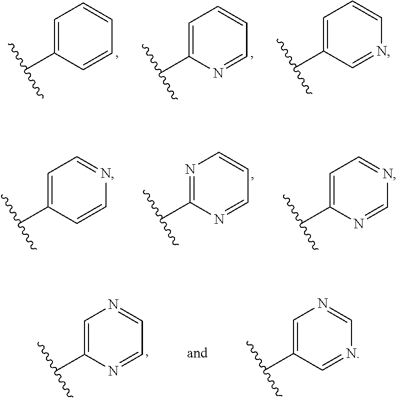

In some embodiments, R¹ is iodine and R² is independently selected from the group consisting of:

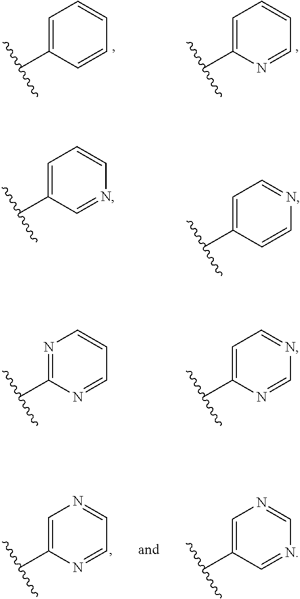

In some embodiments, R² is selected from the group consisting of hydrogen, halogen, —OH, —CN, —CF₃, —NH₂, and C₁₋₆alkyl, and R¹ is independently selected from the group consisting of:

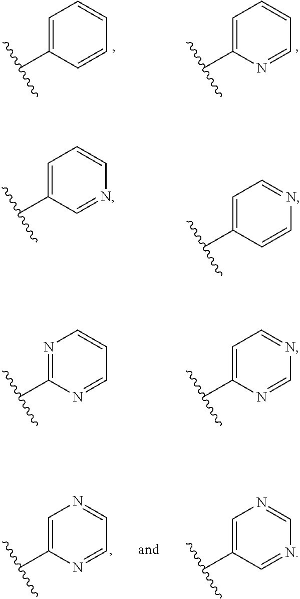

In some embodiments, R² is hydrogen and R¹ is independently selected from the group consisting of:

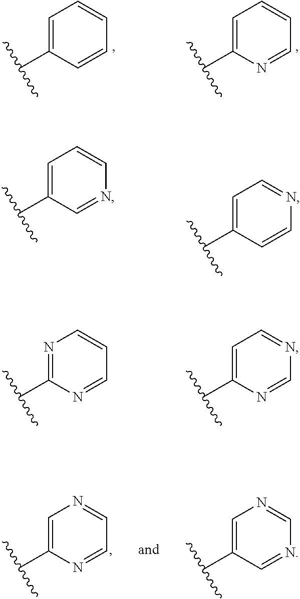

In some embodiments, R² is halogen and R¹ is independently selected from the group consisting of:

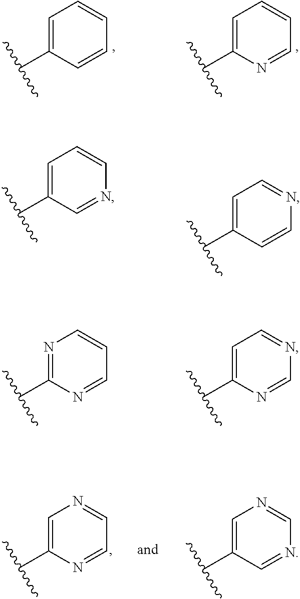

In some embodiments, R² is —OH and R¹ is independently selected from the group consisting of:

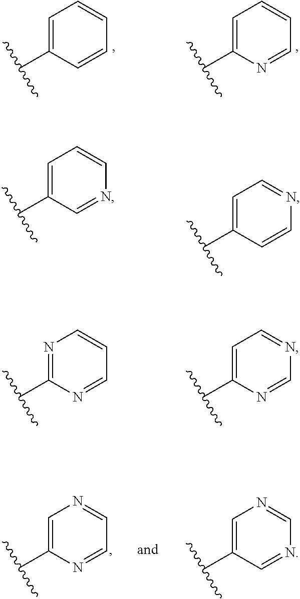

In some embodiments, R² is —CN and R¹ is independently selected from the group consisting of:

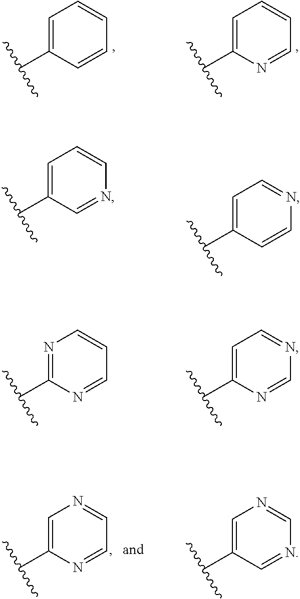

In some embodiments, R² is —CF₃ and R¹ is independently selected from the group consisting of:

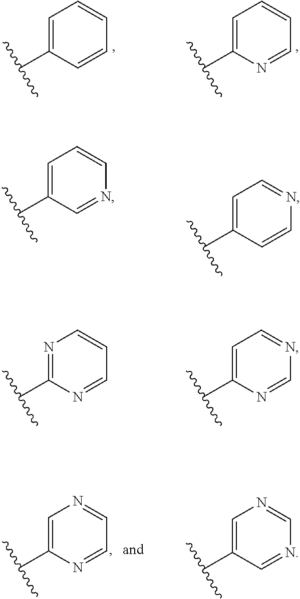

In some embodiments, R² is —NH₂ and R¹ is independently selected from the group consisting of:

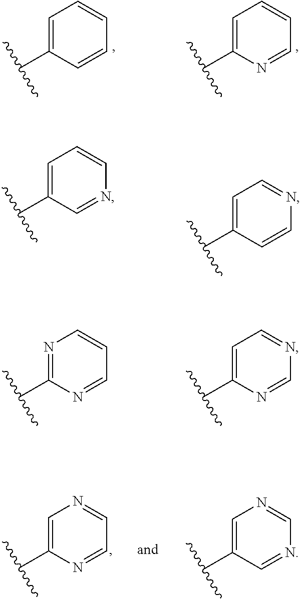

In some embodiments, R² is C₁₋₆alkyl and R¹ is independently selected from the group consisting of:

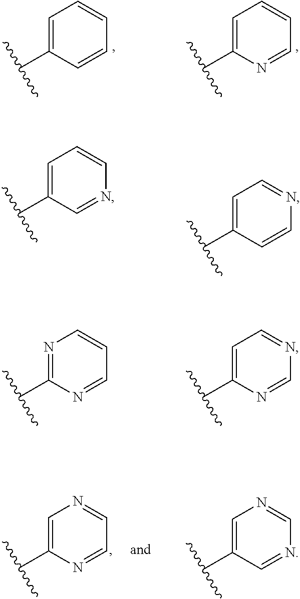

In some embodiments, R² is chlorine and R¹ is independently selected from the group consisting of:

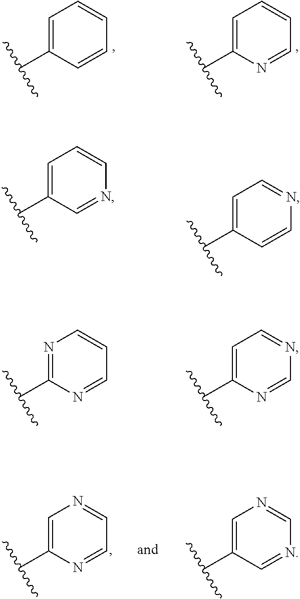

In some embodiments, R² is bromine and R¹ is independently selected from the group consisting of:

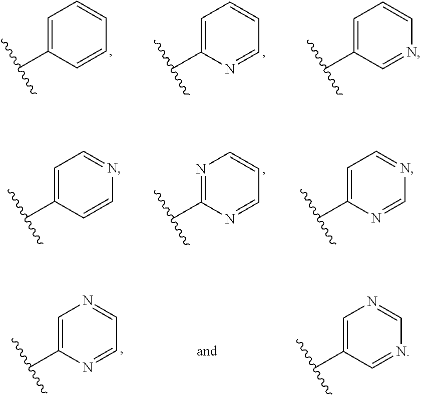

In some embodiments, R² is fluorine and R¹ is independently selected from the group consisting of:

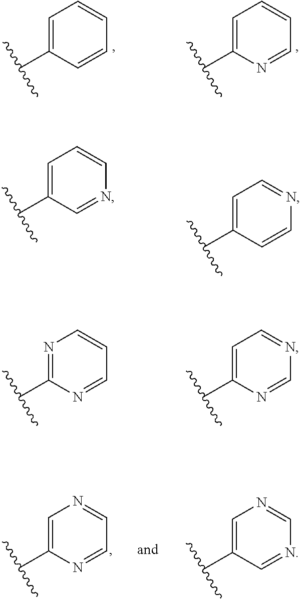

In some embodiments, R² is iodine and R¹ is independently selected from the group consisting of:

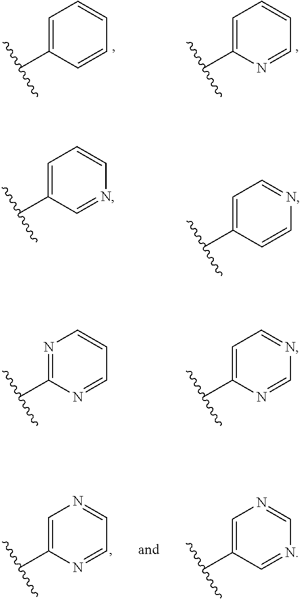

In some embodiments, R¹ and R² are each independently selected from the group consisting of:

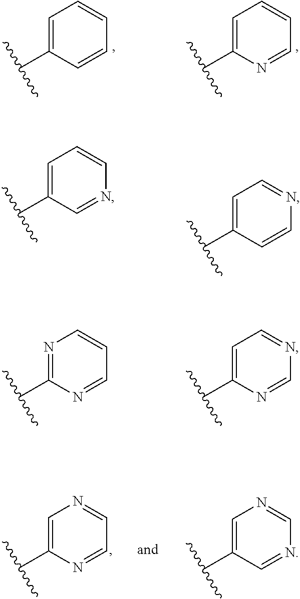

In some embodiments, the compound of Formula I is selected from the group consisting of:

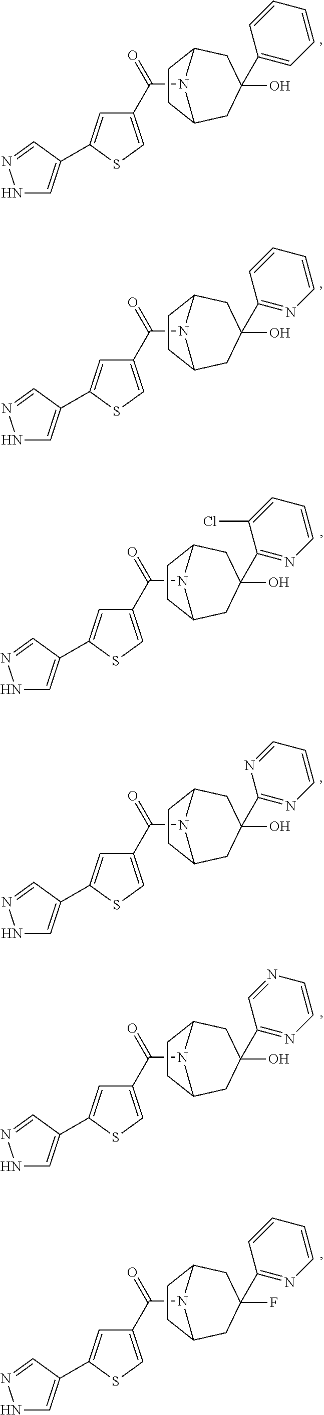 [SMILES: O=C(c1csc(-c2cn[nH]c2)c1)N1C2CCC1CC(O)(c1ccccc1)C2 ; O=C(c1csc(-c2cn[nH]c2)c1)N1C2CCC1CC(O)(c1ccccn1)C2 ; O=C(c1csc(-c2cn[nH]c2)c1)N1C2CCC1CC(O)(c1ncccc1Cl)C2 ; O=C(c1csc(-c2cn[nH]c2)c1)N1C2CCC1CC(O)(c1ncccn1)C2 ; O=C(c1csc(-c2cn[nH]c2)c1)N1C2CCC1CC(O)(c1cnccn1)C2 ; O=C(c1csc(-c2cn[nH]c2)c1)N1C2CCC1CC(F)(c1ccccn1)C2]

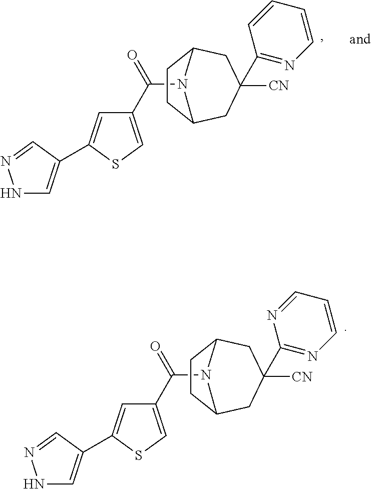 [SMILES: N#CC1(c2ccccn2)CC2CCC(C1)N2C(=O)c1csc(-c2cn[nH]c2)c1 ; N#CC1(c2ncccn2)CC2CCC(C1)N2C(=O)c1csc(-c2cn[nH]c2)c1]

In some embodiments, the compound of Formula I is:

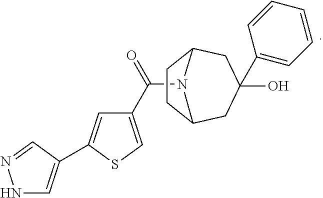 [SMILES: O=C(c1csc(-c2cn[nH]c2)c1)N1C2CCC1CC(O)(c1ccccc1)C2]

In some embodiments, the compound of Formula I is:

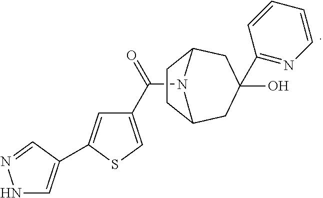 [SMILES: O=C(c1csc(-c2cn[nH]c2)c1)N1C2CCC1CC(O)(c1ccccn1)C2]

In some embodiments, the compound of Formula I is:

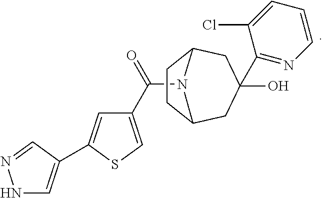 [SMILES: O=C(c1csc(-c2cn[nH]c2)c1)N1C2CCC1CC(O)(c1ncccc1Cl)C2]

In some embodiments, the compound of Formula I is:

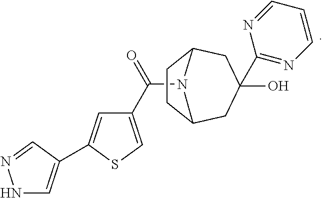 [SMILES: O=C(c1csc(-c2cn[nH]c2)c1)N1C2CCC1CC(O)(c1ncccn1)C2]

In some embodiments, the compound of Formula I is:

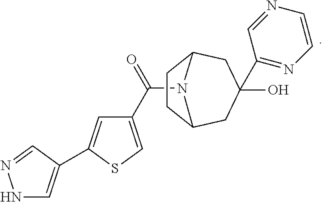 [SMILES: O=C(c1csc(-c2cn[nH]c2)c1)N1C2CCC1CC(O)(c1cnccn1)C2]

In some embodiments, the compound of Formula I is:

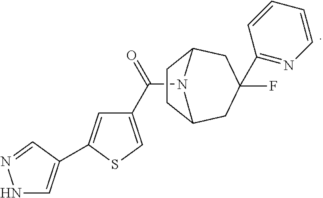 [SMILES: O=C(c1csc(-c2cn[nH]c2)c1)N1C2CCC1CC(F)(c1ccccn1)C2]

In some embodiments, the compound of Formula I is:

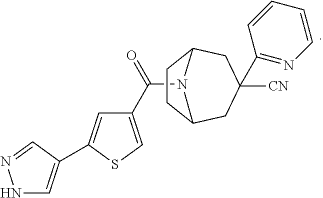 [SMILES: N#CC1(c2ccccn2)CC2CCC(C1)N2C(=O)c1csc(-c2cn[nH]c2)c1]

In some embodiments, the compound of Formula I is:

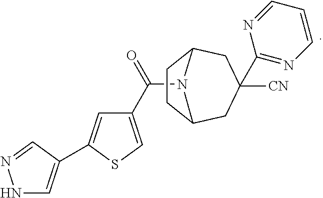 [SMILES: N#CC1(c2ncccn2)CC2CCC(C1)N2C(=O)c1csc(-c2cn[nH]c2)c1]

As would be understood by the person skilled in the art, the compound of Formula I includes any stereoisomers of the depicted structure. That is, the compound of Formula I includes a racemic mixture.

As used herein, a compound of Formula Ia has the following chemical structure:

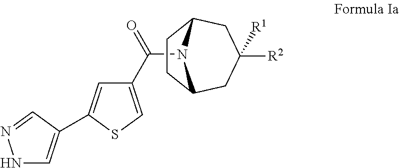

The definitions of R¹ and R² are the same as those provided for a compound of Formula I. That is, the difference between a compound of Formula I and a compound of Formula Ia is that the stereochemistry in a compound of Formula Ia has been resolved.

In some embodiments, the compound of Formula I, or a pharmaceutically acceptable salt, solvate or prodrug thereof, has a particular stereochemistry as depicted in the following chemical structure:

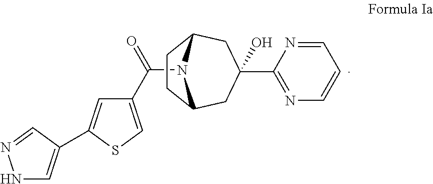

The compound of Formula Ia, when having such particular stereochemistry, is also referred to as “UE2343” or “Xanamem”, and has CAS No.: 1346013-80-6. The chemical name (i.e., IUPAC name) of Formula Ia is (5-(1H-Pyrazol-4-yl)thiophen-3-yl)(3-hydroxy-3-(pyrimidin-2-yl)-8-azabicyclo[3.2.1] octan-8-yl)methanone.

A compound of Formula I, or a pharmaceutically acceptable salt, solvate or prodrug thereof, may be prepared by any suitable method as would be understood by the person skilled in the art. A compound of Formula I, or a pharmaceutically acceptable salt, solvate or prodrug thereof, may be prepared in accordance with the procedure described in WO2011135276. Salts

It may be convenient or desirable to prepare, purify and/or handle a corresponding salt of the compound, such as, for example, a pharmaceutically acceptable salt. As used herein, the term “pharmaceutically acceptable salt” refers to pharmaceutically acceptable organic or inorganic salts. Examples of pharmaceutically acceptable salts are discussed in Berge et al., 1977, “Pharmaceutically Acceptable Salts,” J. Pharm. Sci., vol. 66, p 1-19.

For example, if the compound is anionic, or has a functional group that may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of suitable substituted ammonium ions include, but are not limited to, those derived from ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group that may be cationic (e.g., —NH₂ may be —NH₃⁺), then a salt may be formed with a suitable anion. Examples of suitable inorganic cations include, but are not limited to, those derived from the inorganic acids including hydrochloric, hydrobromic, hydroiodic, sulfuric, sulfurous, nitric, nitrous, phosphoric, and phosphorous. Examples of suitable organic anions include, but are not limited to, those derived from the organic acids including 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicyclic, stearic, succinic, sulfanilic, tartaric, toluenesulfonic, and valeric. Examples of suitable polymeric organic anions include, but are not limited to, those derived from polymeric acids including tannic acid and carboxymethyl cellulose.

A pharmaceutically acceptable salt may involve the inclusion of another molecule such as an acetate ion, a succinate ion or other counterion. The counterion may be any organic or inorganic moiety that stabilises the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport. Unless otherwise specified herein, reference to a particular compound also includes salts thereof.

### Solvates and Hydrates

It may be convenient or desirable to prepare, purify and/or handle a corresponding solvate of the compound. Those skilled in the art of organic chemistry and/or medicinal chemistry will appreciate that many organic compounds can form complexes with solvents in which they are reacted or from which they are precipitated or crystallised. Such complexes are referred to as “solvates”, and as used herein, the term “solvate” refers to such a complex of solute (e.g., a compound, salt of a compound) and solvent. Examples of solvents that may form pharmaceutically acceptable solvates include, but are not limited to, isopropanol, ethanol, methanol, DMSO, ethylacetate, acetic acid, and ethanolamine. If the solvate is water, the solvate may be conventionally referred to as a “hydrate”. In some embodiments, the pharmaceutically acceptable solvate is a pharmaceutically acceptable hydrate. The hydrate may be, for example, a mono-hydrate, a di-hydrate, a tri-hydrate, etc. Unless otherwise specified herein, reference to a particular compound also includes solvates thereof.

### Prodrugs

It may be convenient or desirable to prepare, purify, and/or handle the compound in the form of a prodrug. The term “prodrug”, as used herein, pertains to compound which, when metabolised (e.g., in vivo), yields the desired active compound. Typically, the prodrug is inactive, or less active that the desired active compound, but may provide advantageous handling, administration, or metabolic properties.

Also, as would be understood by a person skilled in the art of organic chemistry and/or medicinal chemistry, some prodrugs are activated enzymatically to yield the active compound, or a compound which, upon further chemical reaction, yields the active compound. For example, the prodrug may be a sugar derivative or other glycoside conjugate, or may be an amino acid ester derivative.

### Methods and Uses

The present disclosure provides for a method of preventing and/or treating cognitive decline in a cognitively healthy subject comprising administering to the cognitively healthy subject a therapeutically effective amount of a compound of Formula I, or a pharmaceutically acceptable salt, solvate or prodrug thereof. The present disclosure also provides for use of a compound of Formula I, or a pharmaceutically acceptable salt, solvate or prodrug thereof, for the prevention and/or treatment of cognitive decline in a cognitively healthy subject. A compound of Formula I, or a pharmaceutically acceptable salt, solvate or prodrug thereof, may be used in the manufacture of a medicament for the prevention and/or treatment of cognitive decline in a cognitively healthy subject.

The present disclosure provides for a method of improving cognition in a cognitively healthy subject comprising administering to the cognitively healthy subject a therapeutically effective amount of a compound of Formula I, or a pharmaceutically acceptable salt, solvate or prodrug thereof. The present disclosure also provides for use of a compound of Formula I, or a pharmaceutically acceptable salt, solvate or prodrug thereof, for improving cognition in a cognitively healthy subject. A compound of Formula I, or a pharmaceutically acceptable salt, solvate or prodrug thereof, may be used in the manufacture of a medicament for improving cognition in a cognitively healthy subject.

It will be appreciated that the dosage regimens and compositions as described herein may apply to any of the embodiments or examples of the methods as described herein.

### Dosage Regimen

As used herein, “therapeutically effective amount” refers to a compound of Formula I, or a pharmaceutically acceptable salt, solvate or prodrug thereof, being administered in an amount sufficient to alleviate or prevent to some extent one or more of the symptoms of the disorder or condition being treated, typically without undue adverse side effects or to achieve a desired pharmacological effect or therapeutic improvement with a reduced side effect profile. The results can be the reduction and/or alleviation of the signs, symptoms, or causes of a disease or condition, or any other desired alteration of a biological system. In some embodiments, the term “therapeutically effective amount” refers to a compound of Formula I, or a pharmaceutically acceptable salt, solvate or prodrug thereof, being administered in an amount sufficient to result in a reduction of symptoms associated with cognitive decline. Therapeutically effective amounts may, for example, be determined by routine experimentation, including but not limited to a dose escalation clinical trial. The phrase “therapeutically effective amount” includes, for example, a prophylactically effective amount. In some embodiments, a prophylactically effective amount is an amount sufficient to prevent cognitive decline. It is understood that “an effective amount” or “a therapeutically effective amount” can vary from subject to subject, due to variation in metabolism of the compound and any of age, weight, general condition of the subject, the condition being treated, the severity of the condition being treated, and the judgment of the prescribing physician. An appropriate an effective amount” or “a therapeutically effective amount” in any individual case may be determined by one of ordinary skill in the art using routine experimentation.

The amount of a compound of Formula I, or a pharmaceutically acceptable salt, solvate or prodrug thereof, that will be effective in the treatment and/or prevention of a particular disorder or condition disclosed herein will depend on the nature of the disorder or condition, and can be determined by standard clinical techniques. In addition, in vitro or in vivo assays may optionally be employed to help identify optimal dosage ranges. Such techniques are known to the person skilled in the art.

The precise dose to be administered to the subject will also depend on the route of administration, and the seriousness of the disease or disorder, and should be decided according to the judgment of the practitioner and each subject's circumstances. For example, suitable dosage ranges for oral administration, are generally from about 0.001 milligram to 1000 milligrams of the compound of Formula I, or a pharmaceutically acceptable salt, solvate or prodrug thereof per kilogram body weight.

In some embodiments, the compound of Formula I, or a pharmaceutically acceptable salt, solvate or prodrug thereof, is administered in an amount so as to deliver a total daily dosage (in mg) of at least about 1, 5, 10, 15, 20, 25, 30, 35, 40, 45, 50, 60, 75, 100, 150, or 200. In some embodiments, the compound of Formula I, or a pharmaceutically acceptable salt, solvate or prodrug thereof, is administered in an amount so as to deliver a total daily dosage (in mg) of less than about 200, 150, 100, 75, 60, 50, 45, 40, 35, 30, 25, 20, 15, 10, 5, or 1. The total daily dosage may be provided in a range between at any two of these upper and/or lower amounts. For example, a total daily dosage may be provided in an amount of between about 1 and 100 mg, about 5 and 75 mg, about 10 and 50 mg, about 15 and 45 mg, or about 20 and 40 mg.

In some embodiments, a therapeutically effective amount of a compound of Formula I, or a pharmaceutically acceptable salt, solvate or prodrug thereof, is administered to the subject at a predetermined frequency. In some embodiments, a compound of Formula I, or a pharmaceutically acceptable salt, solvate or prodrug thereof, is administered to the subject according to a dosage regimen in which a compound of Formula I, or a pharmaceutically acceptable salt, solvate or prodrug thereof is administered once daily, twice daily, three times daily, or four times daily. In some embodiments, the a compound of Formula I, or a pharmaceutically acceptable salt, solvate or prodrug thereof is administered to the subject according to a dosage regimen in which a compound of Formula I, or a pharmaceutically acceptable salt, solvate or prodrug thereof is administered once daily. In some embodiments, a compound of Formula I, or a pharmaceutically acceptable salt, solvate or prodrug thereof is administered to the subject according to a dosage regimen in which a compound of Formula I, or a pharmaceutically acceptable salt, solvate or prodrug thereof is administered twice daily. In some embodiments, a compound of Formula I, or a pharmaceutically acceptable salt, solvate or prodrug thereof is administered to the subject according to a dosage regimen in which a compound of Formula I, or a pharmaceutically acceptable salt, solvate or prodrug thereof is administered three times daily. In some embodiments, a compound of Formula I, or a pharmaceutically acceptable salt, solvate or prodrug thereof is administered to the subject according to a dosage regimen in which a compound of Formula I, or a pharmaceutically acceptable salt, solvate or prodrug thereof is administered four times daily. In some embodiments, a compound of Formula I, or a pharmaceutically acceptable salt, solvate or prodrug thereof is administered to the subject according to a dosage regimen in which a compound of Formula I, or a pharmaceutically acceptable salt, solvate or prodrug thereof is administered multiple times daily. In some examples, a compound of Formula I, or a pharmaceutically acceptable salt, solvate or prodrug thereof is administered as a once daily dose of between about 10 mg and 30 mg, for example at about 20 mg. In other examples, a compound of Formula I, or a pharmaceutically acceptable salt, solvate or prodrug thereof is administered as a twice daily dose of between about 5 mg and 20 mg per dose, for example at about 10 mg per dose. In some embodiments, a compound of Formula I, or a pharmaceutically acceptable salt, solvate or prodrug thereof is administered as a three-times daily dose of between about 5 mg and 15 mg per dose, for example at about 10 mg per dose.

In some embodiments, a therapeutically effective amount of a compound of Formula I, or a pharmaceutically acceptable salt, solvate or prodrug thereof, is administered to the subject at a predetermined frequency and/or duration. For example, administration according to any embodiments (e.g. frequency) as described herein may be for a duration of about, or at least about, 1 day, 1 week, 2 weeks, 3 weeks, 1 month, 2 months, 3 months, 4 months, 5 months, 6 months, 12 months, 2 years, or 5 years. Administration of the therapeutically effective amount of a compound of Formula I, or a pharmaceutically acceptable salt, solvate or prodrug thereof, may be ongoing so long as a therapeutic effect is received by the subject.

As used herein, the term “administer” and “administering” are used to mean introducing the compound of Formula I, or a pharmaceutically acceptable salt, solvate or prodrug thereof, into a subject. When administration is for the purpose of treatment, the compound of Formula I, or a pharmaceutically acceptable salt, solvate or prodrug thereof, is provided at, or after the onset of, a symptom of cognitive decline. The therapeutic administration of this substance serves to attenuate any symptom, or prevent additional symptoms from arising. When administration is for the purposes of preventing or reducing the likelihood cognitive decline, the compound of Formula I, or a pharmaceutically acceptable salt, solvate or prodrug thereof, is provided in advance of any visible or detectable symptom. The prophylactic administration of the compound of Formula I, or a pharmaceutically acceptable salt, solvate or prodrug thereof, serves to attenuate subsequently arising symptoms or prevent or reduce the likelihood of the symptoms from arising altogether.

A compound of Formula I, or a pharmaceutically acceptable salt, solvate or prodrug thereof, may be administered by any suitable route. Examples include, but are not limited to, oral, topical, transdermal, intranasal, vaginal, rectal, intraarterial, intramuscular, intraosseous, intraperitoneal, epidural and intrathecal. In some embodiments, a compound of Formula I, or a pharmaceutically acceptable salt, solvate or prodrug thereof, is administered orally.

A compound of Formula I, or a pharmaceutically acceptable salt, solvate or prodrug thereof, may be administered to the subject with respect to the subject's fasted state, as would be understood by the person skilled in the art. For example, the subject may be administered a compound of Formula I, or a pharmaceutically acceptable salt, solvate or prodrug thereof before, with, or after a meal. In some embodiments, a compound of Formula I, or a pharmaceutically acceptable salt, solvate or prodrug thereof is administered to the subject before a meal (i.e., the subject being in a fasted state). In some embodiments, a compound of Formula I, or a pharmaceutically acceptable salt, solvate or prodrug thereof is administered to the subject with a meal. In some embodiments, a compound of Formula I, or a pharmaceutically acceptable salt, solvate or prodrug thereof is administered at a certain interval (i.e., 30 mins, 1 hour, 2 hours, 3 hours, etc.) following a meal.

### Compositions

Compositions suitable for use in the methods and uses described herein comprise a compound of Formula I, or a pharmaceutically acceptable salt, solvate or prodrug thereof. In some embodiments, a compound of Formula I, or a pharmaceutically acceptable salt, solvate or prodrug thereof, is presented as a composition. In some embodiments, a compound of Formula I, or a pharmaceutically acceptable salt, solvate or prodrug thereof, is presented as a pharmaceutical composition.

The present disclosure also provides pharmaceutical compositions that comprise a compound of Formula I, or a pharmaceutically acceptable salt, solvate or prodrug thereof, with one or more pharmaceutically acceptable carriers, and optionally any other therapeutic ingredients, stabilisers, or the like. The carrier(s) must be pharmaceutically acceptable in the sense of being compatible with the other ingredients of the formulation and not unduly deleterious to the recipient thereof. Generally, suitable pharmaceutically acceptable carriers are known in the art and are selected based on the end use application. The pharmaceutically acceptable carrier may act as a diluent, dispersant or carrier for the active agents and other optional components of the composition. The pharmaceutically acceptable carrier may also contain materials commonly used in pharmaceutically products and can be in a wide variety of forms. For example, the carrier may be water, liquid or solid emollients, silicone oils, emulsifiers, surfactants, solvents, humectants, thickeners, powders, propellants and the like.

In some embodiments, the composition is a pharmaceutical composition, and wherein the composition comprises a pharmaceutically acceptable excipient.

The composition may for example contain a solvent, such as water (e.g. water for injection) or a pharmaceutically acceptable organic solvent.

The compositions may further include diluents, buffers, citrate, trehalose, binders, disintegrants, thickeners, lubricants, preservatives (including antioxidants), inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations).

The compositions of the present disclosure may also include polymeric excipients/additives or carriers, e.g., polyvinylpyrrolidones, derivatised celluloses such as hydroxymethylcellulose, hydroxyethylcellulose, and hydroxypropylmethylcellulose, Ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin.

Other pharmaceutical carriers, excipients, optional ingredients and/or additives suitable for use in the compositions according to the present disclosure are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

A compound of Formula I, or a pharmaceutically acceptable salt, solvate or prodrug thereof, of the present disclosure may be formulated in compositions including those suitable for inhalation to the lung, by aerosol, parenteral (including intraperitoneal, intravenous, subcutaneous, or intramuscular injection) or oral administration.

The compositions may conveniently be presented in unit dosage form and may be prepared by any of the methods well known in the art of pharmacy. All methods include the step of bringing a compound of Formula I, or a pharmaceutically acceptable salt, solvate or prodrug thereof, into association with a carrier that constitutes one or more accessory ingredients.

In general, the compositions are prepared by bringing a compound of Formula I, or a pharmaceutically acceptable salt, solvate or prodrug thereof into association with a liquid carrier to form a solution or a suspension, or alternatively, by bringing a compound of Formula I, or a pharmaceutically acceptable salt, solvate or prodrug thereof into association with formulation components suitable for forming a solid, optionally a particulate product, and then, if warranted, shaping the product into a desired delivery form.

In some embodiments, the composition is formulated for oral delivery. Compositions for oral delivery may, for example, be in the form of tablets, lozenges, aqueous or oily suspensions, granules, powders, emulsions, capsules, syrups, or elixirs. Orally administered compositions may contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in a tablet or pill form, the compositions may be coated to delay disintegration and absorption in the gastrointestinal tract thereby providing a sustained action over an extended period of time. Oral compositions can include standard vehicles such as mannitol, lactose, starch, magnesium stearate, sodium saccharine, cellulose, magnesium carbonate, etc. Such vehicles are preferably of pharmaceutical grade. The oral compositions described herein may contain from about 1% to about 95% of a compound of Formula I, or a pharmaceutically acceptable salt, solvate or prodrug thereof by weight, and the oral compositions may be dosed 1, 2, 3, 4, 5 or more times daily. The oral compositions described herein may contain a compound of Formula I, or a pharmaceutically acceptable salt, solvate or prodrug thereof by weight % in at least about 1, 5, 10, 15, 20, 25, 30, 35, 40, 45, 50, 55, 60, 65, 70, 75, 80, 85, or 90. The oral compositions described herein may contain a compound of Formula I, or a pharmaceutically acceptable salt, solvate or prodrug thereof by weight % in less than about 95, 90, 85, 80, 75, 70, 65, 60, 55, 50, 45, 40, 35, 30, 25, 20, 15, 10, or 5. The oral compositions described herein may contain a compound of Formula I, or a pharmaceutically acceptable salt, solvate or prodrug thereof by weight % in a range provided by any two of these upper and/or lower values, for example between about 5 and 20 wt %.

In some embodiments, the composition is formulated for parenteral delivery. For example, in one embodiment, the composition may be a sterile, lyophilized, crystalized or amorphous composition that is suitable for reconstitution in an aqueous vehicle prior to injection.

In one embodiment, a composition suitable for parenteral administration conveniently comprises a sterile aqueous preparation of a compound of Formula I, or a pharmaceutically acceptable salt, solvate or prodrug thereof, which may for example be formulated to be isotonic with the blood of the recipient.

Pharmaceutical compositions are also provided which are suitable for administration as an aerosol, by inhalation. These formulations comprise a solution or suspension of a compound of Formula I, or a pharmaceutically acceptable salt, solvate or prodrug thereof. The desired formulation may be placed in a small chamber and nebulized. Nebulization may be accomplished by compressed air or by ultrasonic energy to form a plurality of liquid droplets or solid particles comprising a compound of Formula I, or a pharmaceutically acceptable salt, solvate or prodrug thereof.

As discussed below, a compound of Formula I, or a pharmaceutically acceptable salt, solvate or prodrug thereof of the present disclosure may for example be administered in combination with one or more additional pharmaceutically active agents. Thus, in some embodiments, the composition comprises a compound of Formula I, or a pharmaceutically acceptable salt, solvate or prodrug thereof as defined herein, or a pharmaceutically acceptable salt thereof, one or more pharmaceutically acceptable carriers, and one or more additional pharmaceutically active agents, e.g. an additional cognitive enhancing agent.

Generally, the composition comprises a compound of Formula I, or a pharmaceutically acceptable salt, solvate or prodrug thereof in an amount that is therapeutically effective amount. In some embodiments, the therapeutically effective amount is provided by a single dose. In some embodiments, the therapeutically effective amount is provided by one or more doses administered as part of a course of treatment, for example, 1, 2, 3, 4, 5, 6, 7, 8, 9, 10, 11, 12, 13, 14, 15, 16, 17, 18, 19, 20, 21, 22, 23, 24, 25, 26, 27 or greater than 27 doses.

The person skilled in the art would understand that the amount of a compound of Formula I, or a pharmaceutically acceptable salt, solvate or prodrug thereof present in the composition will vary depending on the other ingredients present in the composition, the desired effect and the like. In some embodiments, the composition comprises a compound of Formula I, or a pharmaceutically acceptable salt, solvate or prodrug thereof in a concentration between about 0.001 to 1000 mg/mL, 0.01 to 500 mg/mL, 0.1 to 50 mg/mL. In some embodiments, the composition comprises a compound of Formula I, or a pharmaceutically acceptable salt, solvate or prodrug thereof in a concentration between about 1 to 99 wt %, 1 to 90 wt %, 1 to 85 wt %, 1 to 80 wt %, 1 to 75 wt %, 1 to 70 wt %, 1 to 65 wt %, 1 to 60 wt %, 1 to 55 wt %, 1 to 50 wt %, 1 to 45 wt %, 1 to 40 wt %, 1 to 35 wt %, 1 to 30 wt %, 5 to 99 wt %, 10 to 99 wt %, 15 to 99 wt %, 20 to 99 wt %, 25 to 99 wt %, 30 to 99 wt %, 35 to 99 wt %, 40 to 99 wt %, 45 to 99 wt %, 50 to 99 wt %, 54 to 99 wt %, 60 to 99 wt %, 65 to 99 wt %, 70 to 99 wt %, 75 to 99 wt %, 80 to 99 wt %, 85 to 99 wt %, 90 to 99 wt %, 5 to 90 wt %, 20 to 80 wt %, 30 to 70 wt %, or 40 to 60 wt %.

In some embodiments, a compound of Formula I, or a pharmaceutically acceptable salt, solvate or prodrug thereof may be administered in combination with a further therapeutic agent. In some embodiments, the methods and uses described herein also relate to co-administering one or more substances in addition to a compound of Formula I, or a pharmaceutically acceptable salt, solvate or prodrug thereof to the subject. The term “co-administer” indicates that each of at least two compounds are administered during a time frame wherein the respective periods of biological activity or effects overlap. Thus, the term includes sequential as well as coextensive administration of compounds. Similar to administering compounds, co-administration of more than one substance can be for therapeutic and/or prophylactic purposes. If more than one substance or compound is co-administered, the routes of administration of the two or more substances need not be the same. The scope of the methods and uses described herein are not limited by the identity of the substance or substances which may be co-administered with a compound of Formula I, or a pharmaceutically acceptable salt, solvate or prodrug thereof. For example, compositions comprising a compound of Formula I, or a pharmaceutically acceptable salt, solvate or prodrug thereof may be co-administered with fluids or other substances that are capable of alleviating, attenuating, preventing or removing symptoms in a subject suffering from, exhibiting the symptoms of, or at risk of suffering from cognitive decline. Types of fluid that can be co-administered with a compound of Formula I, or a pharmaceutically acceptable salt, solvate or prodrug thereof should be specific to the circumstances surrounding the particular subject that is suffering from, exhibiting the symptoms of, or at risk of suffering from a bacterial infection. For example, fluids that may be co-administered with a compound of Formula I, or a pharmaceutically acceptable salt, solvate or prodrug thereof include but are not limited to, electrolytes and/or water, salt solutions, such as sodium chloride and sodium bicarbonate, as well as whole blood, plasma, serum, serum albumin and colloid solutions.

The composition may be a food or beverage, or provided into a food or beverage. The composition may be a veterinary product, such as a dog or cat food product. The composition may be a feed additive, a supplement, or a medical food. In some embodiments, a compound of Formula I, or a pharmaceutically acceptable salt, solvate or prodrug thereof, may be incorporated into food products and beverages. The compound of Formula I, or pharmaceutically acceptable salt, solvate or prodrug thereof, may be impregnated, mixed, emulsified, sprayed or coated onto carriers such as cellulose, methylcellulose, dextrose, cyclodextrose, cyclodextrin, maltitol, and fibre. Delivery may also be enhanced with a range of surfactants, lipids, complexes, solvents and co-solvent pharmaceutical delivery systems known in the pharmaceutical art to improve bioavailability, absorption and efficacy. As used herein, the term “food” or “food product” includes any edible product for human or non-human consumption, such as but not limited to supplements, snacks (sweet and savory), cocoa-containing foods, flavours, beverages, dietary supplements and formulations including supplements used in animal health and nutrition. Additional ingredients desired in the resulting food product may be added at any point in the process.

The present disclosure includes food products comprising one or more compounds of Formula I alone as the active ingredient or in combination with other active ingredients.

In some embodiments, there is provided a food product comprising a compound of Formula I, or a pharmaceutically acceptable salt, solvate or prodrug thereof. In some embodiments, there is provided a supplement or feed additive product comprising a compound of Formula I, or a pharmaceutically acceptable salt, solvate or prodrug thereof. In some embodiments, there is provided a beverage comprising a compound of Formula I, or a pharmaceutically acceptable salt, solvate or prodrug thereof.

It will be appreciated that administration of the compound may be provided in various forms depending on the application and subject.

### EXAMPLES

### General: Materials and Methods

Formulations comprising a compound of Formula Ia were tested on human subjects to evaluate the use in preventing and/or treating cognitive decline or improving cognitive ability.

The compound of Formula Ia, as described herein, was synthesised and characterised according to the procedure described in WO2011135276.

The compound of Formula Ia is also known as UE2343 or (5-(1H-pyrazol-4-yl)thiophen-3-yl)(3-hydroxy-3-(pyrimidin-2-yl)-8-azabicyclo[3.2.1]octan-8-yl)methanone. Xanamem™ contains the compound of Formula Ia as an active ingredient.

The test was aimed at assessing if, and to what extent, Xanamem™ 20 mg a day may improve performance in healthy elderly subjects from Baseline (Week 0) to Weeks 2, 4, 6, 8, 10, 12 (End of Treatment) and 16 (Follow-up), as measured by changes in the Cogstate Test Battery. 42 subjects were randomised in which 30 subjects received 20 mg Xanamem™ once a day and 12 subjects received matching placebo once a day.

For each visit, excluding the Screening visit and safety visits (Week 6 and Week 10), subjects arrived at the study site having fasted for at least 10 hours. The subjects took Xanamem™ at site after all the pre-dose blood samples had been taken. Xanamem™ was taken 30 minutes after the start of a meal. On non-study visit days, the subjects took Xanamem™ at the same time each morning with food (30 minutes after the start of the meal).

One capsule of Xanamem™, or the matching placebo, was administered orally with approximately 200 mL of warm water once a day and with food in the morning, to the subjects from Baseline (Week 0) to End of Treatment (Week 12) for a total of 12 weeks.

Xanamem™ was formulated in green and cream-coloured size 3, Coni-Snap shaped gelatin capsules as an excipient blend at a dose of 20 mg. The components in 20 mg Xanamem™ and Placebo used in the test, and their contents and % w/w are shown in Tables 1 and 2, respectively.

### Formulations of Xanamem™ and Placebo

TABLE 1
Xanamem ™ 20 mg formulation

| | Weight | Formulation |
|---|---|---|
| Component | per unit | percentage % w/w |
| UE2343 powder | 20 mg | 10.53 |
| Lactose | 160.5 mg | 84.47 |
| Croscarmellose Sodium | 7.6 mg | 4.00 |
| Talc | 0.95 mg | 0.50 |
| Magnesium Stearate | 0.95 mg | 0.50 |
| Total | 190 mg | 100% |

TABLE 2
Placebo formulation

| | Weight | Formulation |
|---|---|---|
| Component | per unit | percentage % w/w |
| Lactose | 180.5 mg | 95 |
| Croscarmellose Sodium | 7.6 mg | 4.00 |
| Talc | 0.95 mg | 0.50 |
| Magnesium Stearate | 0.95 mg | 0.50 |
| Total | 190 mg | 100% |

To assess the efficacy of Xanamem™ 20 mg once a day in improving cognitive ability in the subjects, the Cogstate Test Battery of the subjects were checked at Baseline (Week 0), Weeks 2, 4, and 8, Week 12 (End of Treatment) and Week 16 (Follow-up).

The Cogstate Test Battery included the following tests: detection test, identification test, one card learning test, one back working memory test, and the continuous paired associate learning test (CPAL; paired associate learning).

### Detection Test

The Cogstate Detection test is a measure of simple reaction time and has been shown to provide a valid assessment of psychomotor function. For this test, the subject pressed a “YES” response key as soon as they detected an event (a card turning face up presented in the centre of the computer screen). The software measured the response time to detect each event.

### Identification Test

The Cogstate Identification test is a measure of choice reaction time and has been shown to provide a valid assessment of visual attention. In this test an event (a card turning face up) occurred in the centre of the computer screen and the subject decided “YES” or “NO” as to whether this event met a predefined and unchanging criterion (i.e. is the color of the card red?). The software measured the speed and accuracy of each response.

### One Card Learning Test

The One Card Learning test is a continuous visual recognition learning test that assesses visual learning within a pattern separation model (Yassa et al., 2010). Theoretical models of the pattern separation model specify that information is organized in orthogonal and distinct non-overlapping representations so that new memories can be stored rapidly without interference. The One Card Learning test has been shown to be a valid test of learning and memory.

In this test, the participant attended to the card in the centre of the screen and responded to the question “have you seen this card before in this test?” If the answer was yes, participants were instructed to press the “YES” button, otherwise they were to press the “NO” button if the answer was no. Normal playing cards were displayed (without joker cards).

In this test, six cards were drawn at random from the deck and were repeated throughout the test. These four cards were interspersed with distractors (non-repeating cards). The test ended after 50 trials, without rescheduling for post-anticipatory correct trials. The primary performance measure for this test was the proportion of correct answers (accuracy), which was normalized using an arcsine square root transformation.

### One Back Working Memory Test

The Cogstate One Back memory test is a valid measure of working memory. On this test the subject was shown a single stimulus in the centre of the computer screen (a card turns face up). The subject decided “YES” or “NO” as to whether the current card matched the card that had been seen on the immediately previous trial. The software measured the speed and accuracy of each response.

### Continuous Paired Associate Learning Test (CPAL; Paired Associate Learning)

The Continuous Paired Associate Learning test is a measure of visual associate memory and uses a well-validated paired associate learning paradigm in which the subject must learn the locations of a number of amoeba-like shapes on the computer screen.

This test consisted of a single amoeboid shape displayed in the centre of the screen surrounded by a number of blue-filled circles. Beneath all but two of the blue spheres were amoeboid shapes, one of which matched the central display; the two remaining circles were distractors. In the exposure phase of the test all of the to-be-remembered pattern-location associations were presented on the computer screen simultaneously.

After a five second delay, a pattern was shown in the central location and this signals that the subject should touch the location in the periphery that contained the same pattern. This process continued until the participant had acknowledged all of the pattern-location associations. The learning phase began with the same test display presented during the exposure phase except that now all of the peripheral locations were shown as blue spheres. One of the patterns presented in the exposure phase was presented in the centre location. With the presentation of this pattern, the subject was required to select the peripheral location where an identical pattern was hidden beneath the blue sphere. This process continued until the correct location of each pattern was found.

Finding the correct location for all patterns in the set was defined as a learning trial. There were six learning trials. A single trial delayed recall condition was available for this test after a 10-30 minute delay. The software recorded each move as an error or as a correct move.

### Timing of Cogstate Test Battery

The timing of the Cogstate Test Battery is shown in Table 3.

TABLE 3
Trials and timing for each test in the cognitive battery

| | N | Maximum | Average |
|---|---|---|---|
| | Trials/ | Time | time |
| Test | Screens | Allowed | Required |
| Subject demographic screen | 1 | Unlimited | ~1 minute |
| Detection test instructions & | 1 | Unlimited | ~1 minute |
| demo | | | |
| Detection test | 35 | 5 minutes | ~2 minutes |
| Identification test instructions | 1 | Unlimited | ~1 minute |
| & demo | | | |
| Identification Test | 30 | 5 minutes | ~2 minutes |
| One Card Learning test | 1 | Unlimited | ~1 minute |
| instructions & demo | | | |
| One Card Learning Test | 80 | 5 minutes | ~4 minutes |
| One Back test instructions & | 1 | Unlimited | ~1 minute |
| demo | | | |
| One Back test | 31 | 5 minutes | ~3 minutes |
| Continuous Paired Associate | 1 | 5 minutes | ~1 minute |
| Learning Test instructions & | | | |
| demo | | | |
| Continuous Paired Associate | 7 | Unlimited | ~7 minutes |
| Learning Test | | | |
| Continuous Paired Associate | 1 | 2 minutes | ~1 minute |
| Learning Test-Delayed | | | |
| Recall | | | |
| Estimated total time cognitive | | | 25 minutes |
| battery | | | |

### Cognitive Outcome Measures

Although each of the cognitive tests to be used yields multiple outcome measures, research by Cogstate identified a set of measures that were optimal for the detection of cognitive change in clinical trials at both the group and individual level.

For each cognitive test, a single outcome measure was selected from each test in the battery to minimize experiment-wise error rates. Each outcome measure was selected because it was shown to be optimal for the detection of change because:
it was drawn from a data distribution that contained only a small probability of floor or ceiling effects and no restriction in the range of possible performance values; and
it was drawn from a distribution that is normal or which can be corrected to normal through the use of appropriate mathematical transformation (e.g., logarithmic base 10, or arcsine).
The outcome measures for the test used are summarized in Table 4.

TABLE 4
Recommended outcome measures for the cognitive battery

| | | | Range | |
|---|---|---|---|---|
| | Cognitive | Unit of | of | |
| Test name | domain | measurement | scores | Description |
| Detection | Psychomotor | Log₁₀ | 0 to | Speed of |
| Test | function | milliseconds | 3.69* | performance; |
| | | | | mean of |
| | | | | the log₁₀ |
| | | | | transformed |
| | | | | reaction |
| | | | | times for |
| | | | | correct |
| | | | | responses |
| Identi- | Visual | Log₁₀ | 0 to | Speed of |
| fication | attention | milliseconds | 3.69* | performance; |
| Test | | | | mean of |
| | | | | the log₁₀ |
| | | | | transformed |
| | | | | reaction |
| | | | | times for |
| | | | | correct |
| | | | | responses |
| One Card | Visual | Arcsine | 0-1.57 | Accuracy of |
| Learning | learning | proportion | | performance; |
| Test | | correct | | arcsine |
| | | | | transformation |
| | | | | of the |
| | | | | proportion of |
| | | | | correct |
| | | | | responses |
| One Back | Working | Log₁₀ | 0-3 69 | Speed of |
| Test | memory | milliseconds | | performance: |
| | | | | mean of |
| | | | | the log₁₀ |
| | | | | transformed |
| | | | | reaction |
| | | | | times for |
| | | | | correct |
| | | | | responses |
| Continuous | Hippocampal | Number of | 0-999 | Number of |
| Paired | dependent | errors across | | errors |
| Associate | learning and | all learning | | remembering |
| Learning | memory | trials | | the |
| Test | | | | associations |
| Continuous | Hippocampal | Number of | 0-999 | Number of |
| Paired | dependent | errors across | | errors |
| Associate | learning and | single | | remembering |
| Learning | memory | delayed | | the |
| Test- | | trial | | associations |
| Delayed | | | | after a delay |
| Recall | | | | |

*Reaction times longer than 5 seconds (i.e. log10 [5000]) were excluded as reflecting responses that were abnormally slow.
# for this test total n errors in a single administration was controlled so that patients who did not understand the test were not tested unnecessarily.

The outcomes of each of the Tests included in the Cogstate Test Battery are described below.

### Example 1: Detection Test

Compared to the placebo subject group, the subject group administered Xanamem™ showed improved psychomotor function. This is demonstrated by the improved speed of performance of the subjects administered Xanamem™, which was measured by means of the log₁₀ transformed reaction times for correct responses by the subjects.

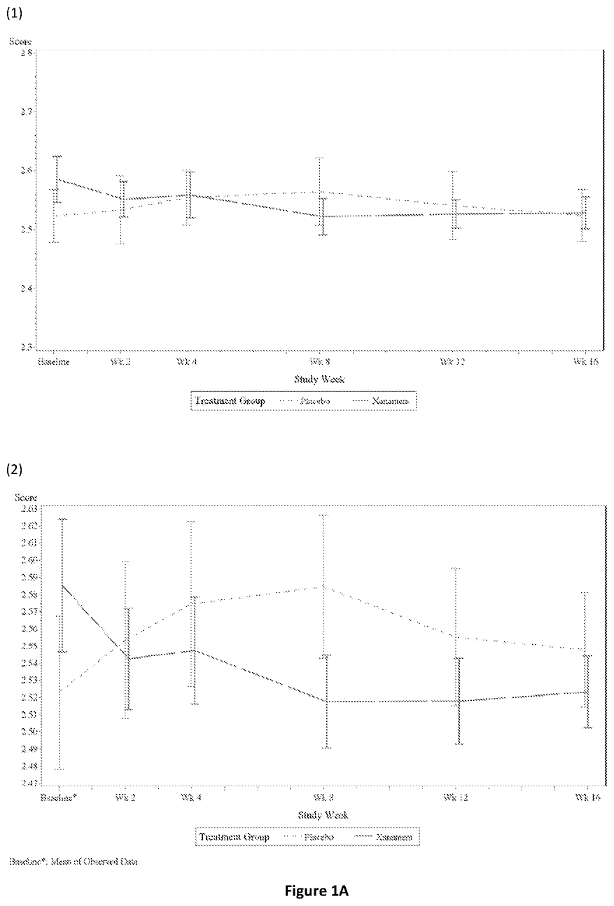

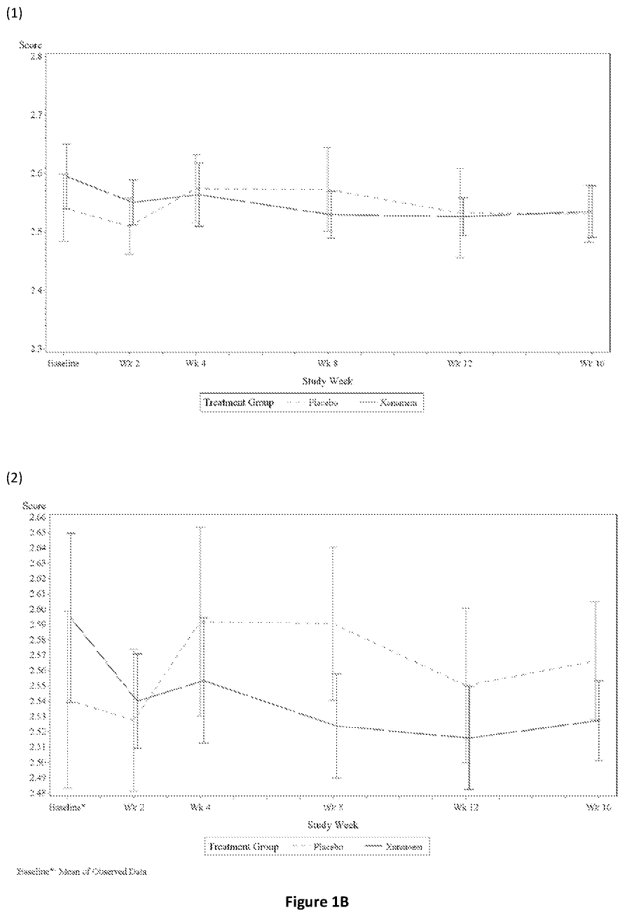

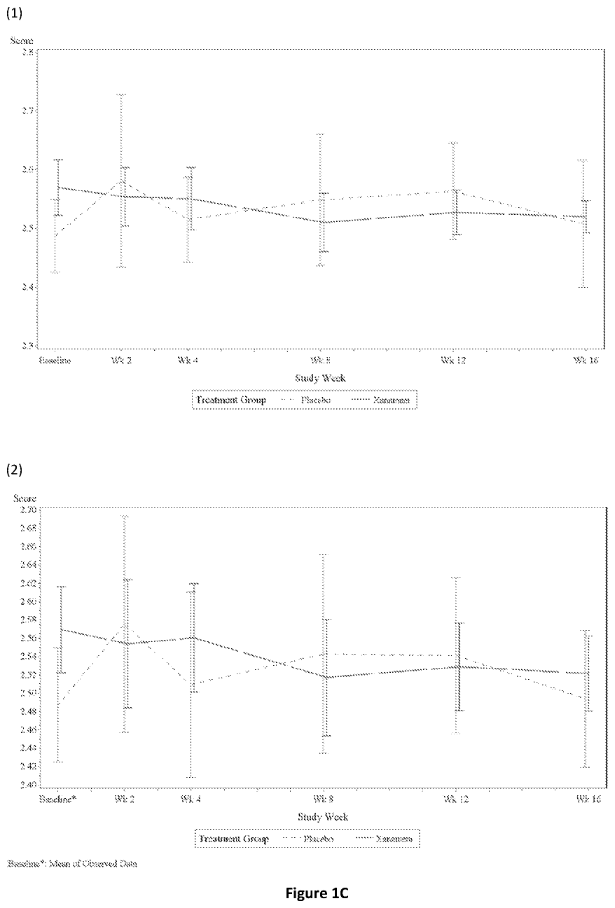

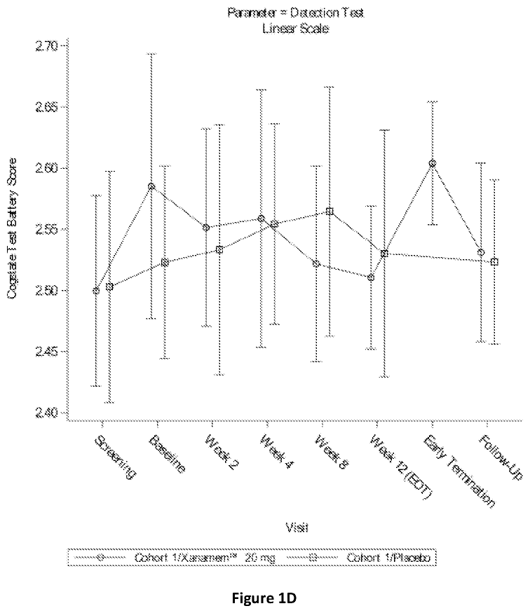

The test scores from the Detection Test are shown in FIG. 1A(1) and FIG. 1A(2) for all of the subjects, FIG. 1B(1) and FIG. 1B(2) for all of the female subjects, and FIG. 1C(1) and FIG. 1C(2) for all of the male subjects. A linear plot of mean (±SD) Detection Test results over visit by treatment group is shown in FIG. 1D.

### Example 2: Identification Test

Compared to the placebo subject group, the subject group administered Xanamem™ showed improved visual attention. This is demonstrated by the improved speed of performance of the subjects administered Xanamem™, which was measured by means of the log₁₀ transformed reaction times for correct responses by the subjects.

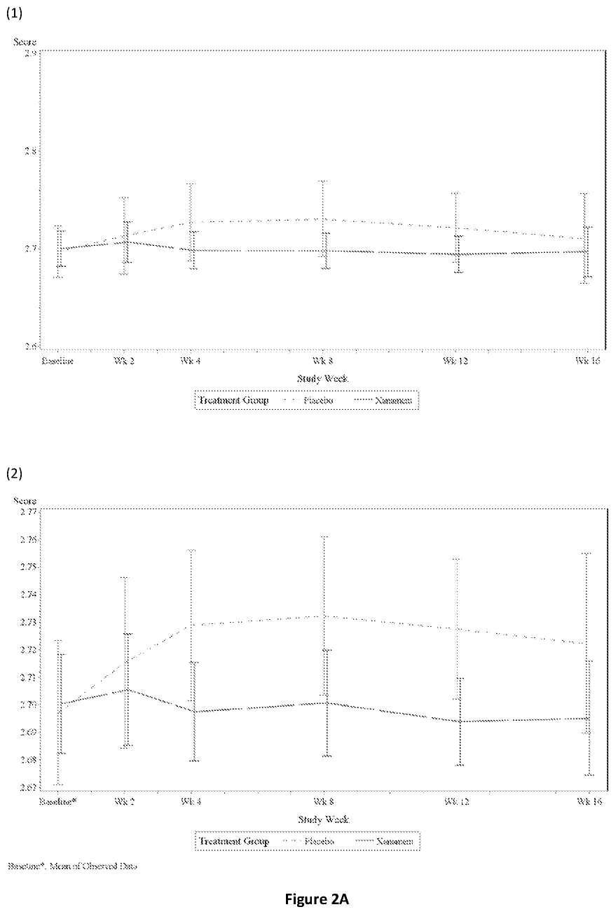

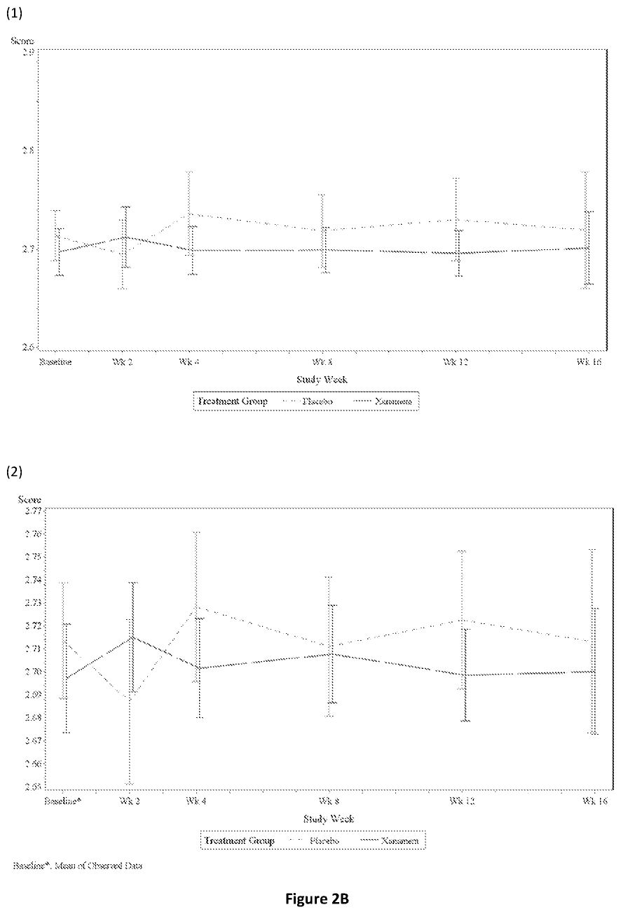

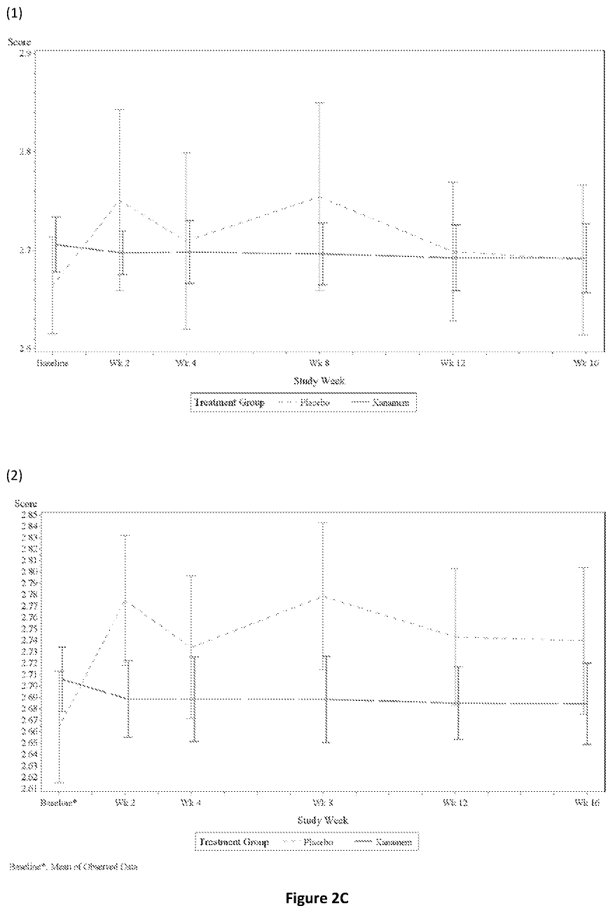

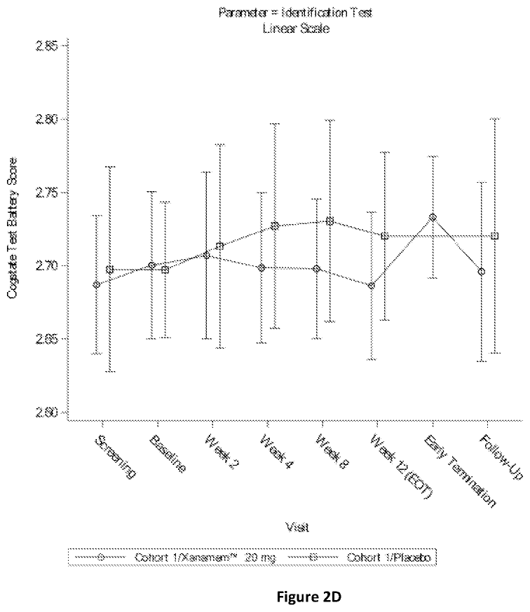

The test scores from the Identification Test are shown in FIG. 2A(1) and FIG. 2A(2) for all of the subjects, FIG. 2B(1) and FIG. 2B(2) for all of the female subjects, and FIG. 2C(1) and FIG. 2C(2) for all of the male subjects. A linear plot of mean (±SD) Identification Test results over visit by treatment group is shown in FIG. 2D.

### Example 3: One Card Learning Test

Compared to the placebo subject group, the subject group administered Xanamem™ showed improved visual learning in week 8. This is demonstrated by the improved accuracy of performance in week 8 of the subjects administered Xanamem™, which was measured by arcsine transformation of the proportion of correct responses by the subjects.

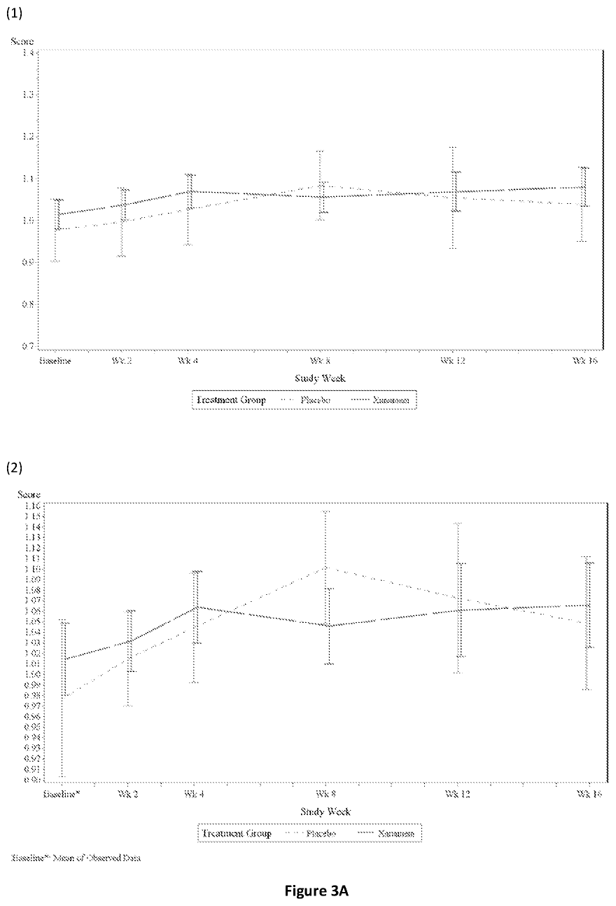

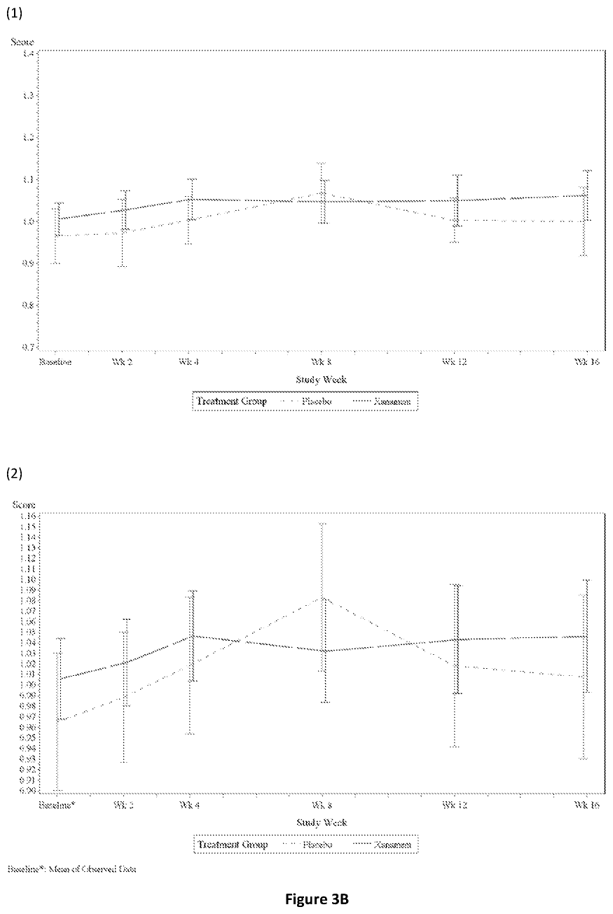

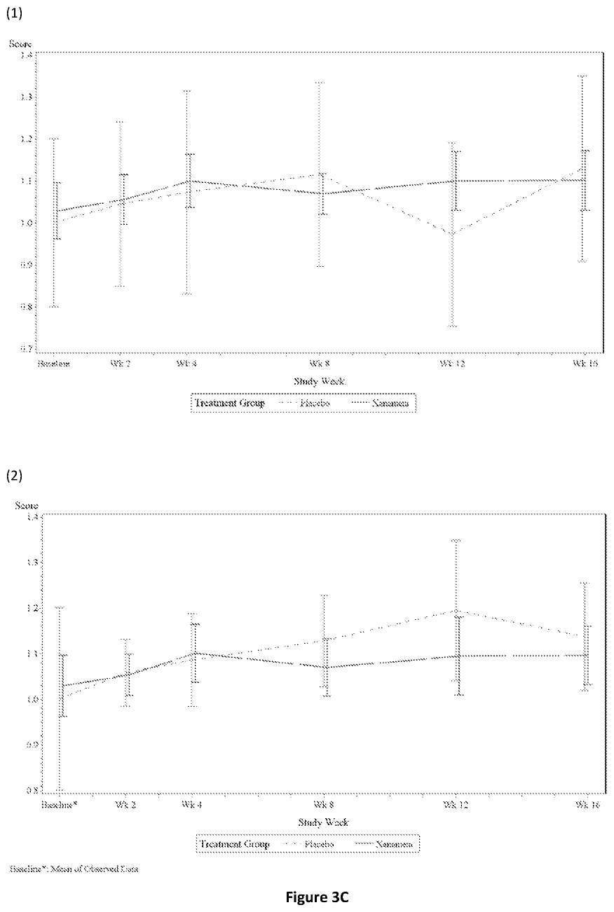

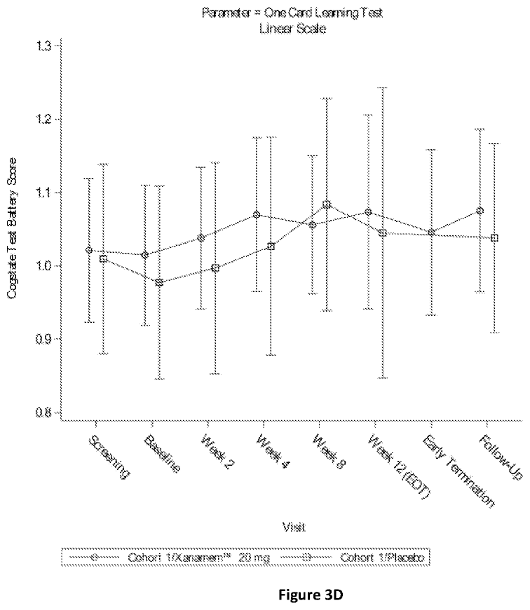

The test scores from the One Card Learning Test are shown in FIG. 3A(1) and FIG. 3A(2) for all of the subjects, FIG. 3B(1) and FIG. 3B(2) for all of the female subjects, and FIG. 3C(1) and FIG. 3C(2) for all of the male subjects. A linear plot of mean (±SD) One Card Learning Test results over visit by treatment group is shown in FIG. 3D.

### Example 4: One Back Working Memory Test

Compared to the placebo subject group, the subject group administered Xanamem™ showed improved working memory. This is demonstrated by the significantly improved speed of performance of the subjects administered Xanamem™, which was measured by means of the log₁₀ transformed reaction times for correct responses by the subjects.

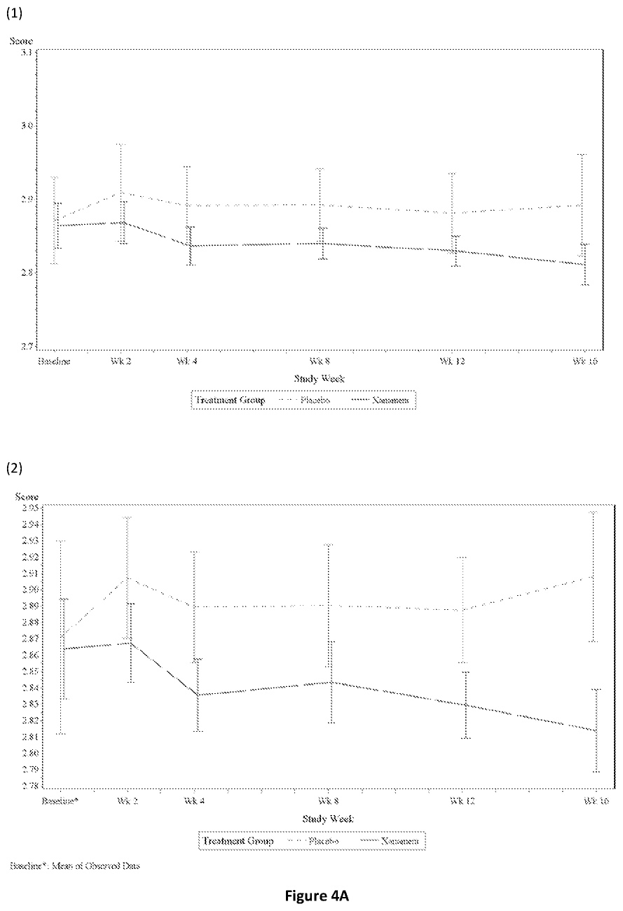

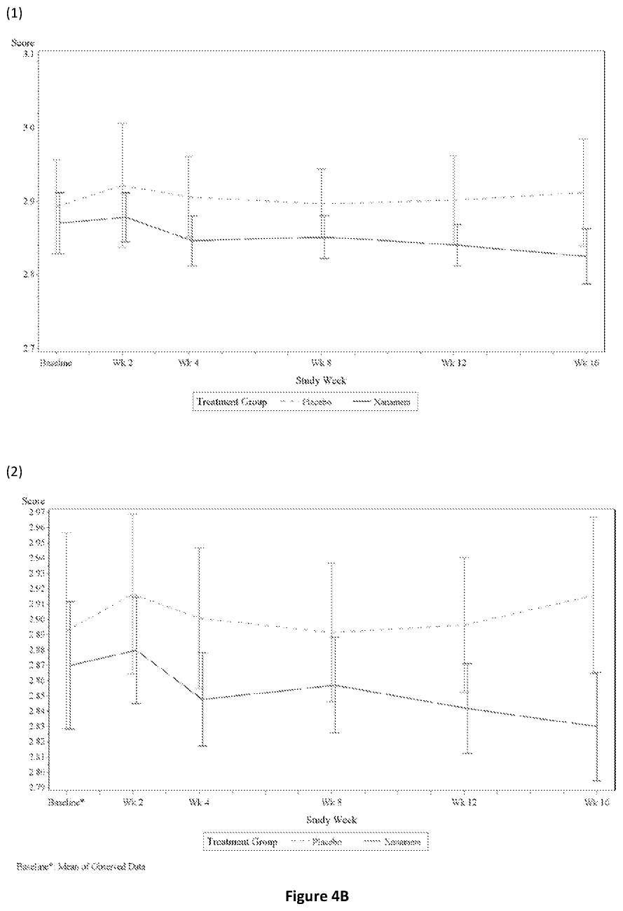

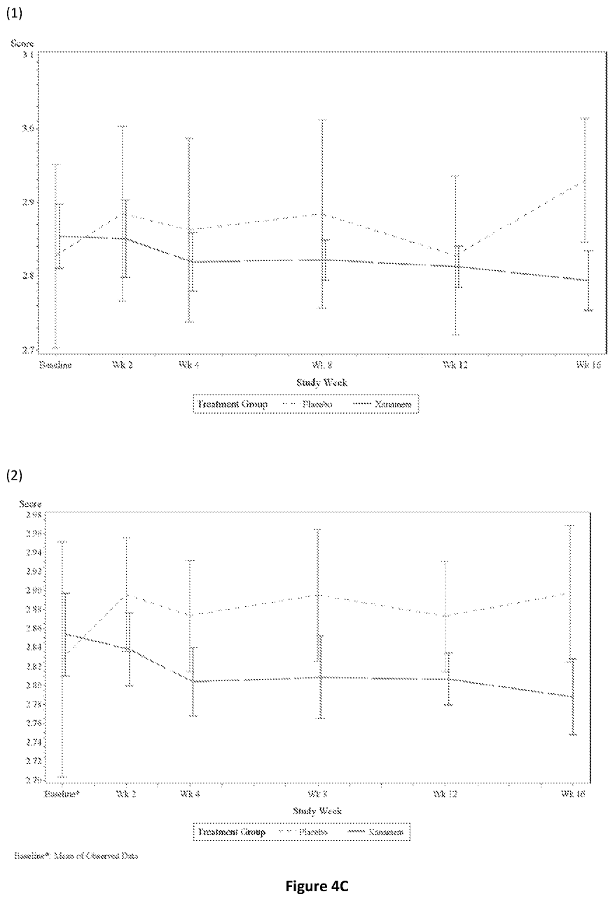

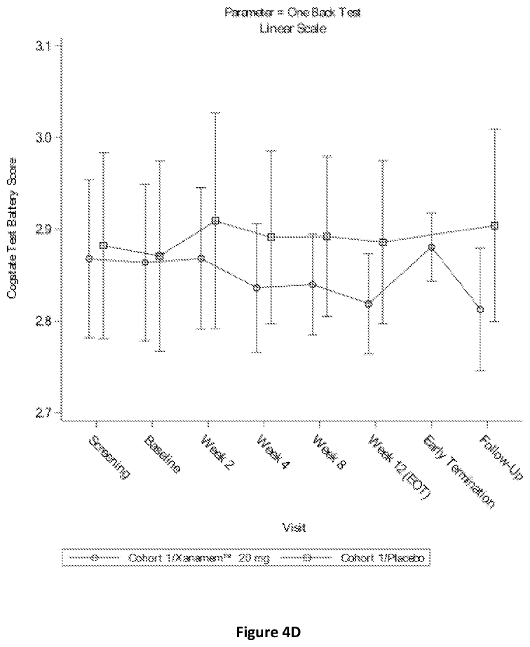

The test scores from One Back Working Memory test are shown in FIG. 4A(1) and FIG. 4A(2) for all of the subjects, FIG. 4B(1) and FIG. 4B(2) for all of the female subjects, and FIG. 4C(1) and FIG. 4C(2) for all of the male subjects. A linear plot of mean (±SD) One Back Test results over visit by treatment group is shown in FIG. 4D.

### Example 5: Continuous Paired Associate Learning Test

Compared to the placebo subject group, the subject group administered Xanamem™ showed improved Hippocampal dependent learning and memory. This is demonstrated by the improved performance of the subjects administered Xanamem™, which was measured by a number of errors remembering the associations by the subjects.

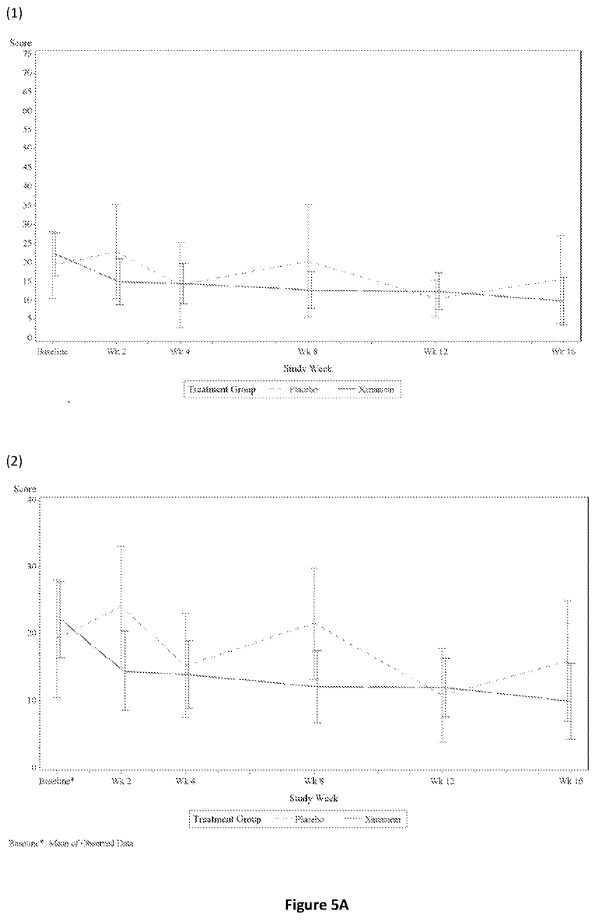

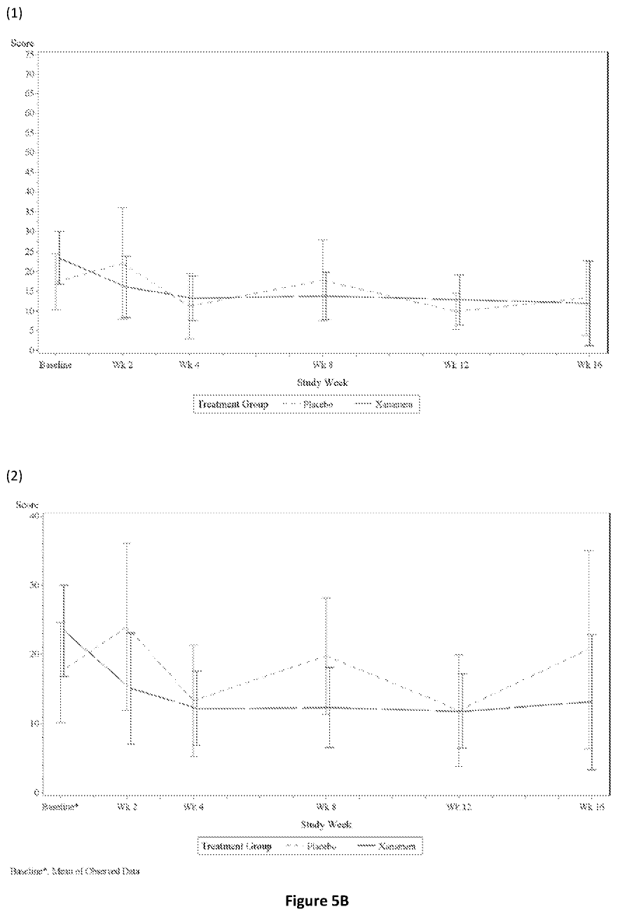

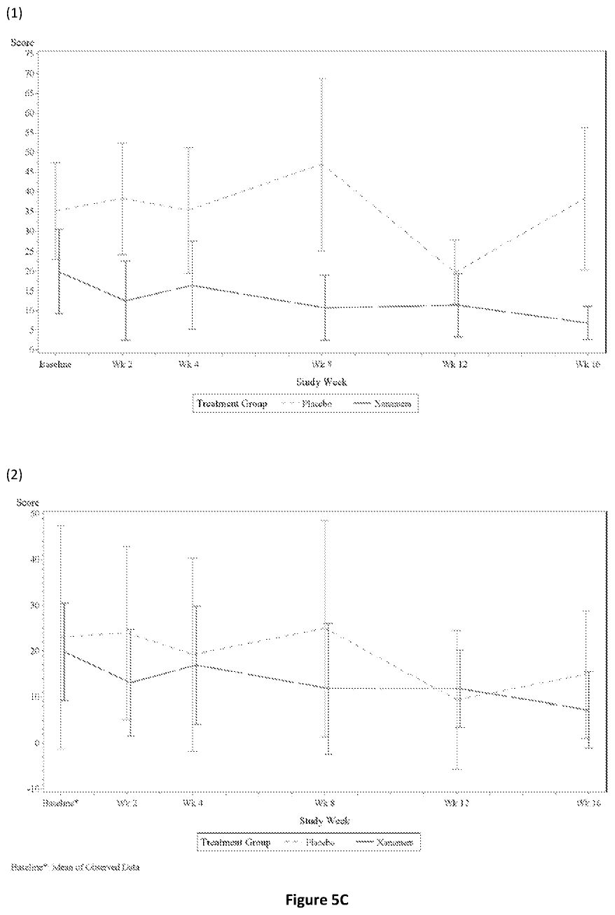

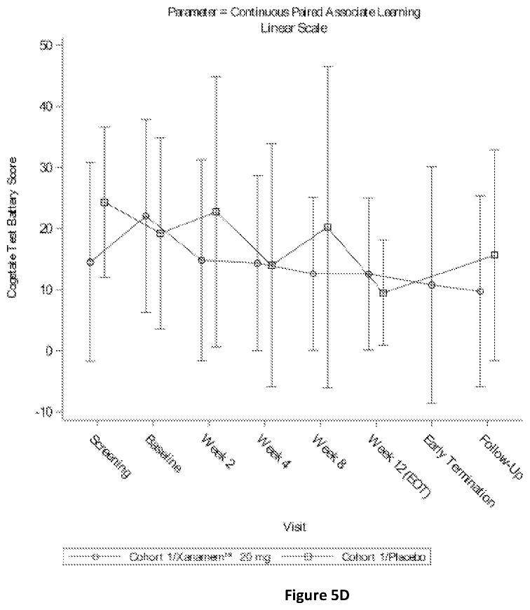

The test scores from the Continuous Paired Associate Learning Test are shown in FIG. 5A(1) and FIG. 5A(2) for all of the subjects, FIG. 5B(1) and FIG. 5B(2) for all of the female subjects, and FIG. 5C(1) and FIG. 5C(2) for all of the male subjects. A linear plot of mean (±SD) Continuous Paired Associated Learning Test results over visit by treatment group is shown in FIG. 5D.

### Example 6: Continuous Paired Associate Learning-Delayed Test

Compared to the placebo subject group, the subject group administered Xanamem™ showed improved Hippocampal dependent learning and memory. This is demonstrated by the improved performance of the subjects administered Xanamem™, which was measured by number of errors remembering the associations after a delay by the subjects.

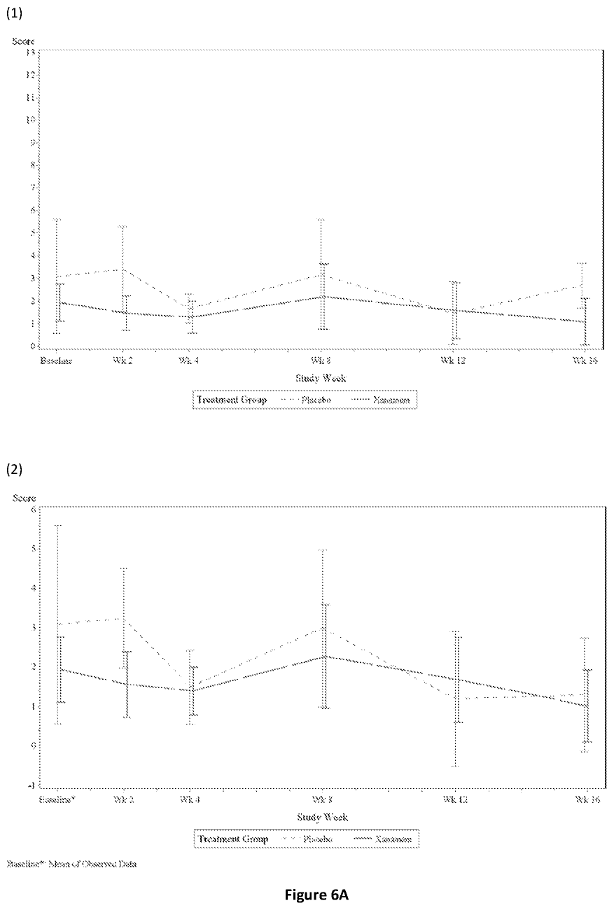

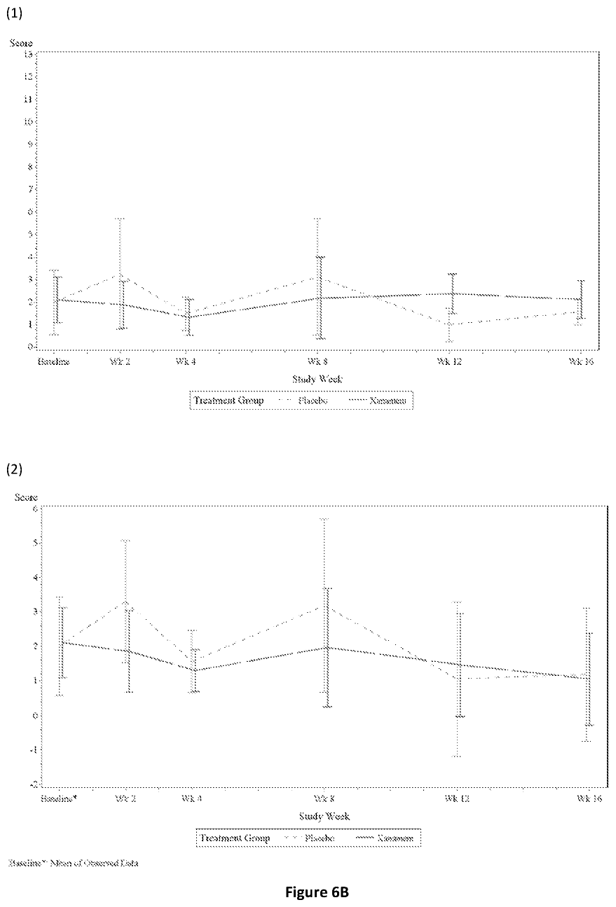

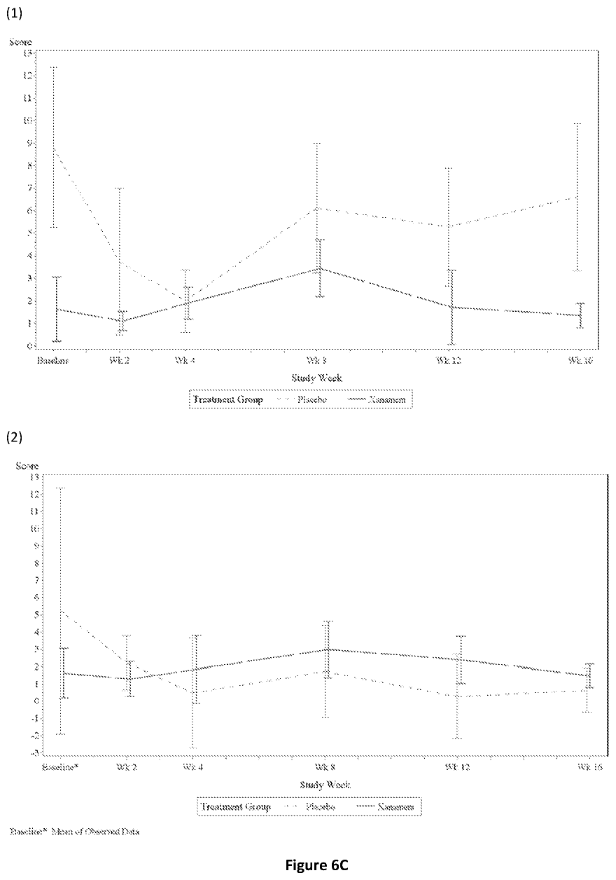

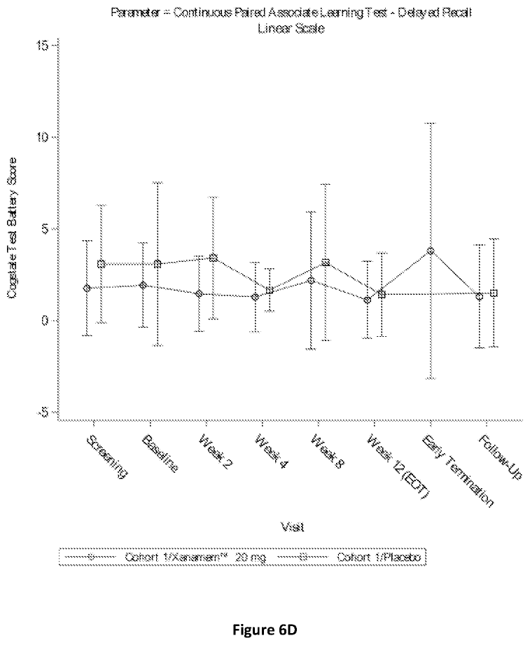

The test scores from the Continuous Paired Associate Learning-Delayed Test are shown in FIG. 6A(1) and FIG. 6A(2) for all of the subjects, FIG. 6B(1) and FIG. 6B(2) for all of the female subjects, and FIG. 6C(1) and FIG. 6C(2) for all of the male subjects. A linear plot of mean (±SD) Continuous Paired Associated Learning-Delayed Test results over visit by treatment group is shown in FIG. 6D.

### Example 7: Conclusions of Cogstate Test Battery

Statistically significant and large effects (>0.6) of 12 weeks treatment with Xanamem™ 20 mg versus placebo were observed for speed of performance on both the Identification and One-back tests, which measure attention and working memory, respectively. These positive effects were evident both at Week 12 (EOT) and also at the earlier post-Baseline assessment time points. Together, they suggest a beneficial effect on cognition of Xanamem™ 20 mg when compared to placebo, and include reducing or preventing performance decrements over time in healthy, elderly subjects. Further, support for this was evident in the data for the Detection test, which measures psychomotor speed. The positive effect from Xanamem™ 20 mg was evident at week 8, and this may have been due, at least in part, to performance differences at Baseline, through effect sizes in favour of Xanamem™ 20 mg were evident at all time points in sensitivity analysis.

Other additionally statistically significant differences in data were in favour of Xanamem™ 20 mg on the Continuous Paired Associates Learning Test outcomes. The effect size difference also favoured Xanamem™ 20 mg at Week 4 in the sensitivity analysis. Overall, the effect size (ES) data for One Card Learning and Continuous Paired Associates outcomes tended to favour Xanamem™ 20 mg.

Importantly, no systemic evidence of impairment in any respect of cognition was observed to be associated with treatment with Xanamem™ 20 mg when compared to placebo over the 12 weeks of this study.

### Example 8: Pharmacokinetics

The mean Xanamem™ plasma concentration at 3-5 hours after the first dose of the study was 169 ng/mL. Trough and 3-5 hour concentrations were consistent across Weeks 2, 4, and 8, suggesting that steady state had been achieved by Week 2. The mean trough concentration at Weeks 2, 4, and 8, was 89, 73, and 78 ng/mL, and the mean 3 to 5 hour concentration was 353. 331, and 330 ng/mL for weeks 2, 4, and 8, respectively.

The ratio of Xanamem™ CSF to plasma concentrations in the four CSF samples taken at the EOT visit ranged from 0.028 to 0.421; the range of CSF concentrations was 3.48 to 6.25 ng/mL, compared to 11.6 to 126 ng/mL for the corresponding plasma concentrations.

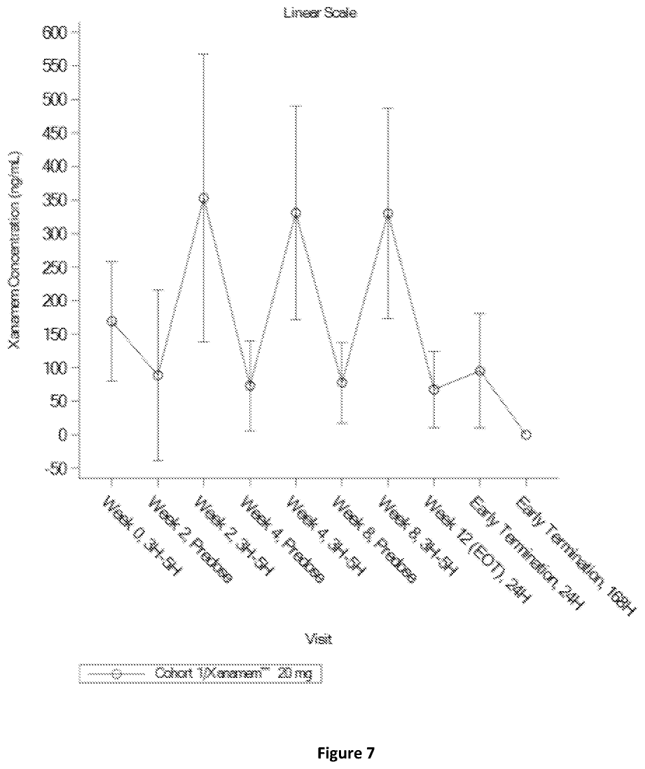

The pharmacokinetics of Xanamem™ were well described using a one-compartment model with first-order absorption and elimination. Only body weight was identified as a covariate impacting PK in this model, confirming that the PK of Xanamem™ are consistent between healthy volunteers, patients with Alzheimer's Disease, and healthy elderly subjects. The results are shown in FIG. 7.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method of preventing and/or treating cognitive decline in a cognitively healthy subject, comprising administering to the cognitively healthy subject a therapeutically effective amount of a compound of Formula I, or a pharmaceutically acceptable salt, solvate or prodrug thereof: 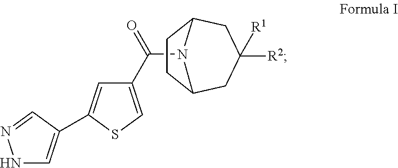
wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR³, —NR³R⁴, —COR³, —CO₂R³, —CONR³R⁴, —NR³COR⁴, —SO₂R³, —SO₂NR³R⁴, and —NR³SO₂R⁴;
wherein R³ and R⁴ are each independently selected from the group consisting of hydrogen, C₁₋₆alkyl, 3-7-membered carbocyclyl and 3-7-membered heterocyclyl;
wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl.

2. (canceled)

3. The method of claim 1, wherein R¹ and R² are each independently selected from the group consisting of hydrogen, halogen, 3-10-membered carbocyclyl, 3-10-membered heterocyclyl, —OH, —CN, and —NH₂; and wherein if present, each 3-10-membered carbocyclyl and 3-10-membered heterocyclyl may be further substituted with one or more substituents selected from the group consisting of hydrogen, halogen, —OH, —CN, —CF₃, —NH₂, and C₁₋₆alkyl.

4. The method of claim 1, wherein R¹ and R² are each independently selected from the group consisting of hydrogen, halogen, 6-membered carbocyclyl, 6-membered heterocyclyl, —OH, —CN, and —NH₂; and wherein if present, each 6-membered carbocyclyl and 6-membered heterocyclyl may be further substituted with one or more substituents selected from the group consisting of hydrogen, halogen, —OH, —CN, —CF₃, —NH₂, and C₁₋₆alkyl.

5. The method of claim 1, wherein R¹ is selected from the group consisting of hydrogen, halogen, —OH, —CN, —CF₃, —NH₂, and C₁₋₆alkyl, and R² is independently selected from the group consisting of:

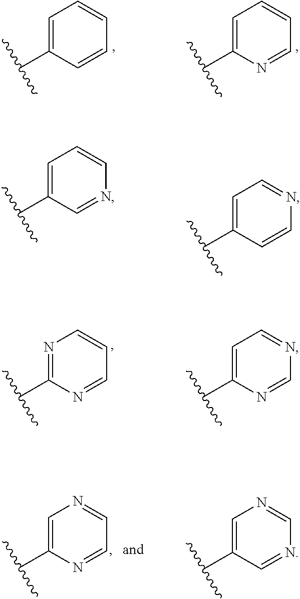

6. The method of claim 1, wherein the compound of Formula I is selected from the group consisting of:

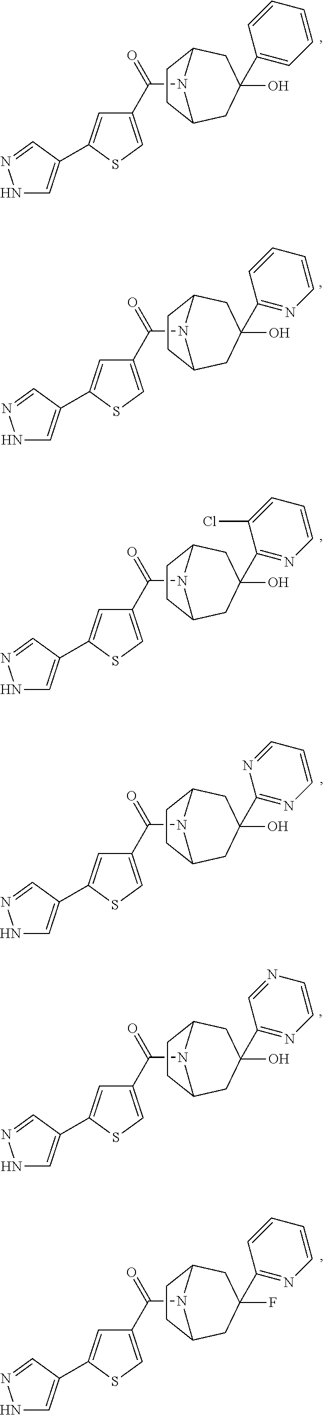 [SMILES: O=C(c1csc(-c2cn[nH]c2)c1)N1C2CCC1CC(O)(c1ccccc1)C2 ; O=C(c1csc(-c2cn[nH]c2)c1)N1C2CCC1CC(O)(c1ccccn1)C2 ; O=C(c1csc(-c2cn[nH]c2)c1)N1C2CCC1CC(O)(c1ncccc1Cl)C2 ; O=C(c1csc(-c2cn[nH]c2)c1)N1C2CCC1CC(O)(c1ncccn1)C2 ; O=C(c1csc(-c2cn[nH]c2)c1)N1C2CCC1CC(O)(c1cnccn1)C2 ; O=C(c1csc(-c2cn[nH]c2)c1)N1C2CCC1CC(F)(c1ccccn1)C2]

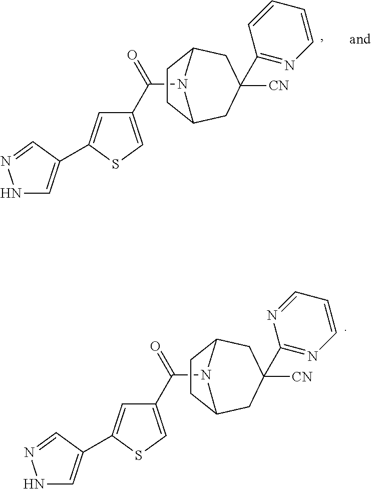 [SMILES: N#CC1(c2ccccn2)CC2CCC(C1)N2C(=O)c1csc(-c2cn[nH]c2)c1 ; N#CC1(c2ncccn2)CC2CCC(C1)N2C(=O)c1csc(-c2cn[nH]c2)c1]

7. The method of claim 1, wherein the compound of Formula I is:

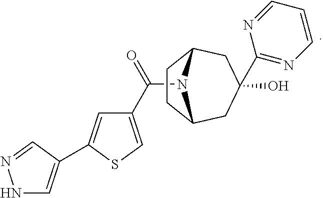 [SMILES: O=C(c1csc(-c2cn[nH]c2)c1)N1[C@@H]2CC[C@H]1C[C@](O)(c1ncccn1)C2]

8. (canceled)

9. The method of claim 1, wherein the cognitively healthy subject is not suffering from another, diagnosed medical condition that is associated with the central nervous system (CNS) and the subject has an increased risk of cognitive decline.

10. (canceled)

11. The method of claim 9, wherein the increased risk of cognitive decline is provided by a risk factor selected from the group consisting of an increased risk of stroke, the presence of genetic markers associated with cognitive decline, a family history of cognitive decline, environmental factors associated with cognitive decline, societal factors associated with cognitive decline, external factors associated with cognitive decline, and side effects associated with therapeutic treatment.

12. The method of claim 1, wherein the administration of a compound of Formula I, or a pharmaceutically acceptable salt, solvate or prodrug thereof, provides improved cognitive ability of the treated subject relative to a non-treated subject determinable by cognitive testing.

13. (canceled)

14. The method of claim 1, wherein the administration of a compound of Formula I, or a pharmaceutically acceptable salt, solvate or prodrug thereof, provides an improvement of psychomotor function of the treated subject relative to a non-treated subject.

15. The method of claim 14, wherein the improvement of psychomotor function is at least partial restoration of motor function provided by an improvement in simple reaction time.

16. (canceled)

17. The method claim 1, wherein the administration of a compound of Formula I, or a pharmaceutically acceptable salt, solvate or prodrug thereof, provides an improvement of visual attention of the treated subject relative to a non-treated subject.

18. The method of claim 17, wherein the improvement in visual attention is provided by an improvement in choice retention time.

19. The method of claim 1, wherein the administration of a compound of Formula I, or a pharmaceutically acceptable salt, solvate or prodrug thereof, provides an improvement of learning and memory of the treated subject relative to a non-treated subject, by an improvement in visual recognition learning.

20. (canceled)

21. The method of claim 1, wherein the administration of a compound of Formula I, or a pharmaceutically acceptable salt, solvate or prodrug thereof, provides an improvement of working memory of the treated subject relative to a non-treated subject.

22. The method of claim 1, wherein the administration of a compound of Formula I, or a pharmaceutically acceptable salt, solvate or prodrug thereof, provides an improvement of visual associate memory of the treated subject relative to a non-treated subject.

23. The method of claim 1, wherein the cognitively healthy subject is selected from one or more of the following categories;
has age-related cognitive decline;
is at least 40 years of age, or between about 1 and about 20 years of age;
has a low IQ;
is male.

24-27. (canceled)

28. The method of claim 1, wherein the compound of Formula I, or a pharmaceutically acceptable salt, solvate or prodrug thereof, is administered in amount so as to deliver a total daily dosage of from about 20 to 40 mg of Formula I, administered as a single once-daily dosage or a twice-daily dosage.

29. (canceled)

30. The method of claim 1, wherein the compound of Formula I, or a pharmaceutically acceptable salt, solvate or prodrug thereof, is administered as a twice-daily dosage of 10 mg per dosage.

31. The method of claim 1, wherein the compound of Formula I, or a pharmaceutically acceptable salt, solvate or prodrug thereof, is administered orally.

32-38. (canceled)
